SEQUENCE LISTING (2 sequences)

SEQ ID NO: 1 - RNA, 22 nt, Artificial Sequence
  Synthetic miR-16 mimic, hsa-mir-16-1
ccaguauuaacugugcugcuga

SEQ ID NO: 2 - RNA, 22 nt, Artificial Sequence
  Synthetic miR-34a mimic, hsa-mir-34a
uggcagugucuuagcugguugu

(19) United States
(12) Patent Application Publication
(10) Pub. No.: US 2020/0171067 A1
(43) Pub. Date: Jun. 4, 2020

(54) Cancer Treatment Based on Delivery of Oligoes via Gap Junctions from Human Mesenchymal Stem Cells (hMSC)

(71) Applicants: THE TRUSTEES OF COLUMBIA UNIVERSITY IN THE CITY OF NEW YORK, New York, NY (US); THE RESEARCH FOUNDATION FOR THE STATE UNIVERSITY OF NEW YORK, Albany, NY (US)

(72) Inventors: Ira S. COHEN, Stony Brook, NY (US); Peter R. BRINK, Setauket, NY (US); Michael R. ROSEN, New York, NY (US)

(73) Assignees: The Trustees of Columbia University in the City of New York, New York, NY (US); The Research Foundation for the State University of New York, Albany, NY (US)

(21) Appl. No.: 16/087,788

(22) PCT Filed: Mar. 23, 2017
(86) PCT No.: PCT/US2017/023803

§ 371 Date: Sep. 24, 2018

Related U.S. Application Data
(60) Provisional application No. 62/312,230, filed on Mar. 23, 2016.

(51) Int. Cl. A61K 31/713; A61K 31/7105; A61K 35/28; C12N 15/113

(52) U.S. Cl. CPC A61K 31/713; A61K 31/7105; C12N 2320/32; C12N 15/113; C12N 2310/141; A61K 35/28

(57) ABSTRACT

A method of treating cancer in vivo includes introducing in vitro into human mesenchymal stem cells (hMSCs) at least one type of inhibitory oligonucleotide, and contacting a tumor tissue of syncytial cancer cells with the hMSCs in vivo under conditions permitting a hMSC to form a gap junction channel with a first syncytial cancer cell of the tumor tissue. As a result, the at least one type of inhibitory oligonucleotide is delivered into the first syncytial cancer cell by traversing the gap junction channel and the at least one type of inhibitory oligonucleotide is delivered into a second syncytial cancer cell of the tumor tissue by traversing a gap junction channel between the first syncytial cancer cell and the second syncytial cancer cell.

### RELATED APPLICATIONS

This application claims benefit of Provisional Appln. 62/312,230, filed Mar. 23, 2016, the entire contents of which are hereby incorporated by reference as if fully set forth herein, under 35 U.S.C. § 119(e).

This application is related to: U.S. application Ser. No. 10/583,369 filed 17 Dec. 2004 as PCT Application PCT/US2004/042504 which issued as U.S. Pat. No. 7,842673 on 30 Nov. 2010; and, to U.S. Continuation application Ser. No. 12/910,346 filed 22 Oct. 2010 which issued as U.S. Pat. No. 8,188,062 on 29 May 2012.

### STATEMENT OF GOVERNMENT INTEREST

This invention was made with government support under GM055263 awarded by the National Institutes of Health. The government has certain rights in the invention.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “20170323_15003087PC0_ST25.txt” created on Mar. 23, 2017 and is 2 KB in size. The sequence listing contained in this .txt file is part of the specification and is hereby incorporated by reference herein in its entirety

### BACKGROUND OF THE INVENTION

Throughout this application, various publications are referenced within footnotes or in the text within parentheses. Each of these publications in their entireties are hereby incorporated by reference into this application, except for terminology that is inconsistent with that used herein, to more fully describe the state of the art to which this invention pertains. Full bibliographic citations for these references may be found at the end of the specification, preceding the claims.

As described in commonly owned prior application U.S. Ser. No. 10/342,506, filed Jan. 15, 2003, and in Plotnikov et al., 2003, and Valiunas et al., 2002, stem cells have been used to form gap junctions with target tissues. Such stem cells can influence the activity of the target tissues by delivering gene products or small molecules.

As described in U.S. Pat. Nos. 7,842673 and 8,188,062, oligonucleotides, either single or double stranded, or both, can be passed through gap junctions formed by connexin protein Cx43 or connexin protein Cx40 in HeLa cell pairs, as demonstrated by a single electrode delivery of fluorescent-tagged oligonucleotides to a donor cell and determining their transfer to the target cell via gap junction mediated communication. Accordingly, those patents suggest delivery of oligonucleotides to target cells using any donor cell that forms gap junctions.

However, nucleotides in the form of antisense RNA, or siRNA, have not been shown to be efficacious to reduce tumor growth when delivered by human mesenchymal stem cells (hMSCs) to target tissues made up of tumor cells of various cancers. One reason for this is the observation that hMSCs growing together with tumor cells promote vasculature that increases the rate of tumor growth. For example, Tian et al., 2011, states “we found that the promoting role of hMSCs on tumor growth was related with the increase of tumor vessel formation. Our present study suggests that hMSCs have a contradictory effect on tumor cell growth between in vitro and in vivo, and therefore, the exploitation of hMSCs in new therapeutic strategies should be cautious under the malignant conditions.”

### SUMMARY OF THE INVENTION

As described herein, inhibitory oligonucleotide can be passed through gap junctions from hMSCs to tumor tissue in amounts that, surprisingly, are effective at retarding tumor growth in vivo, and are therefore therapeutic. The hMSC serves not only to effectively introduce the inhibitory oligonucleotide into the tumor cells but also reduces exposure of cells outside the tumor to the inhibitory oligonucleotide because the inhibitory oligonucleotide is not introduced systemically but, instead, is introduced locally at a site of contact inside the tumor.

According to a first set of embodiments, a method of treating cancer in vivo includes introducing in vitro into human mesenchymal stem cells (hMSCs) at least one type of inhibitory oligonucleotide, and contacting a tumor tissue comprising syncytial cancer cells with the hMSCs in vivo under conditions permitting a hMSC to form a gap junction channel with a first syncytial cancer cell of the tumor tissue. As a consequence, the inhibitory oligonucleotide is delivered into the first syncytial cancer cell by traversing the gap junction channel and the inhibitory oligonucleotide is delivered into a second syncytial cancer cell of the tumor tissue by traversing a gap junction channel between the first syncytial cancer cell and the second syncytial cancer cell.

Embodiments provides useful treatments in which down regulation of gene activity in certain tumors caused by certain inhibitory oligonucleotide is sufficient to overcome accelerated tumor growth normally observed in the presence of hMSCs.

As compared to prior methods wherein delivery of RNA or antisense to target cells is done by a naked plasmid or by interstitial fluids, in various embodiments the delivery is via hMSC directly into the cytoplasm of cells of the target tissue via gap junctions, and the transfection rate is anticipated to be much higher. Furthermore, in the target tumor cells, the inhibitory oligonucleotide delivered to one tumor cell is transfected to neighboring tumor cells also via gap junctions, at a rate sufficient for retarding growth of the tumor in vivo. Thus, various embodiments provide treatments for certain various cancers.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings in which like reference numerals refer to similar elements and in which:

FIG. 1A shows an example 12 member single stranded oligonucleotide passing through gap junction channels composed of connexin 43, according to an embodiment;

FIG. 1B shows an example 16 member single stranded oligonucleotide passing through gap junction channels composed of connexin 43, according to an embodiment;.

FIG. 1C shows an example 24 member single stranded oligonucleotide passing through gap junction channels composed of connexin 43, according to an embodiment;

FIG. 1D shows an example 12 mer hybridized double stranded oligonucleotide passing through gap junction channels composed of connexin 43, according to an embodiment;.

FIG. 2A is a graph that illustrates a summary of the example data where the x-axis is the length of the oligonucleotide, and the y-axis is the relative intensity of the fluorescent tag in the recipient cell (the cell on the left in all of the examples of FIG. 1A through FIG. 1D) 12 minutes after delivery of the oligonucleotide to the source cell, according to an embodiment;

FIG. 2B is a graph that illustrates example junctional conductance on the x-axis versus relative intensity of the fluorescent tag on the y-axis, according to an embodiment;

FIG. 3A and FIG. 3B are graphs that illustrate example increase in tumor growth rate when treated in vivo with human mesenchymal stem cells (hMSCs);

FIG. 4 is a diagram that illustrate microRNA that interferes with various cell process pathways; and, thus represent potential agents for retarding tumor growth, according to an embodiment;

FIG. 5A through FIG. 5C are plots that illustrate relative effects on tumor growth of various syncytial cancers by potential agents for retarding tumor growth transfected directly in vitro; and thus indicate candidate agents for introduction via hMSCs, according to an embodiment;

FIG. 6, is a plot that illustrates relative effects on melanoma tumor growth by potential agents, including miR-16, for retarding tumor growth transfected directly in vitro; and thus indicate candidate agents for introduction via hMSCs, according to an embodiment;

FIG. 7A through FIG. 7C are plots that illustrate relative effects on prostate tumor growth by potential agents, including SiRNA mimic for microRNA miR-16, for retarding tumor growth transfected directly in vitro; and thus indicate candidate agents for introduction via hMSCs, according to an embodiment;

FIG. 8A through FIG. 8D are images and plots that illustrate relative effects on pancreatic tumor growth by potential agents, including miR-16 mimic and Kras^{GAT} SiRNA, for retarding tumor growth transfected directly in vitro; and thus indicate candidate agents for introduction via hMSCs, according to an embodiment;

FIG. 9A is an image and FIG. 9B is a plot that both illustrate relative effects on pancreatic tumor growth of a different cell line by potential agents, including miR-16 mimic, for retarding tumor growth transfected directly in vitro; and thus indicate candidate agents for introduction via hMSCs, according to an embodiment;

FIG. 10A through FIG. 10F are images and plots that illustrate loading of potential agents for retarding tumor growth transfected directly in vitro into hMSCs, according to an embodiment;

FIG. 11 is a set of plots that illustrate various methods for loading of potential agents for retarding tumor growth transfected directly in vitro into hMSCs, according to various embodiments;

FIG. 12A and FIG. 12B are plots that illustrate survival of hMSCs after loading by potential agents for retarding tumor growth transfected directly in vitro into hMSCs, according to an embodiment;

FIG. 13A and FIG. 13B are plots that illustrate formation of gap junctions between an hMSC and a syncytial cancer cell, according to an embodiment;

FIG. 14A and FIG. 14B illustrate example formation of gap junctions between two syncytial cancer cells for use in propagating an inhibitory oligonucleotide through multiple cells of a syncytial cancer tumor, according to an embodiment;

FIG. 14C and FIG. 14D are images of electrophoresis gels that illustrate gap junction connexins are found in a variety of colorectal cancer cell lines, for use in various embodiments;

FIG. 14E and FIG. 14F are images of electrophoresis gels that illustrate RNA that interferes with the production of several structural or functional proteins can be transfected between cancer cells, for use in various embodiments;

FIG. 15A through FIG. 15C are images and plots that illustrate propagation of siRNA through multiple cells of a syncytial cancer tumor, according to an embodiment;

FIG. 16, is a plot that illustrates relative effects on colorectal tumor growth by co-culture with hMSCs in vitro; according to an embodiment;

FIG. 17, is a plot that illustrates direct relationship between tumor weight and tumor volume for comparing various remaining plots;

FIG. 18A and FIG. 18B are plots that illustrate relative effects on prostate tumor growth by co-culture in vitro with hMSCs loaded with miR-16 or a siRNA mimic for miR-16; according to an embodiment; and,

FIG. 19A through FIG. 19C are plots that illustrate an example effect on prostate tumor growth by in vivo treatment with hMSCs loaded with an siRNA mimic for miR-16; according to an embodiment.

### DETAILED DESCRIPTION

### 1. Definitions

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of skill in the art, such as the Oxford Dictionary of Biochemistry and Molecular Biology (Ed. Anthony Smith, Oxford University Press, Oxford, 2004).

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of +−10% of the referenced value or a precision implied by the smallest non-zero digit.

The term “DNA” or “deoxyribonucleic acid” as used herein means a molecule made up of certain nucleic acid bases. DNA can carry most of the genetic instructions used in the development, functioning and reproduction of all known living organisms and many viruses. DNA is a nucleic acid; alongside proteins and carbohydrates, nucleic acids compose the three major macromolecules essential for all known forms of life. Most DNA molecules consist of two biopolymer strands coiled around each other to form a double helix. The two DNA strands are known as polynucleotides since they are composed of simpler units called nucleic acid bases, or more simply, nucleotides. Each nucleotide is composed of a nitrogen-containing nucleobase—either cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T, and C with G), hydrogen bonds bind the nitrogenous bases of the two separate polynucleotide strands to make double-stranded DNA.

The term “RNA” or “ribonucleic acid” as used herein means a polymeric molecule, often implicated in various biological roles in coding, decoding, regulation, and expression of genes. RNA, like DNA, is a nucleic acid. RNA is a linear molecule composed of four types of smaller molecules called ribonucleotide bases: adenine (A), cytosine (C), guanine (G), and, in place of thymine (T) found in DNA, uracil (U).

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

The term “antisense” as used herein means a sequence of nucleotides complementary to and therefore capable of binding to a coding sequence, which may be either that of the strand of a DNA double helix that undergoes transcription, or that of a messenger RNA molecule. Antisense DNA is the non-coding strand complementary to the coding strand in double-stranded DNA. The antisense strand serves as the template for messenger RNA (mRNA) synthesis.

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides.

The term “hybridization” means hydrogen bonding, which may be Watson-Crick, Hoogsteen or reversed Hoogsteen hydrogen bonding, between complementary nucleoside or nucleotide bases in one or more nucleotides.

The term “synthetic nucleic acid” means that the nucleic acid does not have a chemical structure or sequence of a naturally occurring nucleic acid. Synthetic nucleotides include an engineered nucleic acid such as a DNA or RNA molecule. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. Consequently, it will be understood that the term “synthetic miRNA” refers to a “synthetic nucleic acid” that functions in a cell or under physiological conditions as a naturally occurring miRNA.

The term “oligonucleotide” as used herein means a short DNA or RNA molecule Oligonucleotides readily bind, in a sequence-specific manner, to their respective complementary oligonucleotides, DNA, or RNA to form duplexes.

As used herein, the term “isolated nucleotide” means an nucleotide that is altered or removed from the natural state through human intervention.

The term “mRNA” or “messenger RNA” as used herein means the template for protein synthesis via translation and is a large family of RNA molecules that convey genetic information from DNA to the ribosome. where they specify the amino acid sequence of the protein products of gene expression.

The term small interfering RNA (siRNA), sometimes known as short interfering RNA or silencing RNA, is a class of double-stranded RNA molecules, 20-25 base pairs in length. Various siRNA plays many roles, but it is most notable in the RNA interference (RNAi) pathway, where it interferes with the expression of specific genes with complementary nucleotide sequences. An siRNA functions by causing mRNA to be broken down after transcription, resulting in no translation into a protein. An siRNA that prevents translation to a particular protein is indicated by the protein name coupled with the term siRNA. Thus an siRNA that interferes with the translation to the important kinase Akt is indicated by the expression “Akt siRNA.” Typically, an siRNA in various embodiments is a double-stranded nucleic acid molecule comprising two nucleotide strands, each strand having about 19 to about 28 nucleotides (i.e. about 19, 20, 21, 22, 23, 24, 25, 26, 27, or 28 nucleotides).

The term micro RNA (abbreviated miRNA) is a small non-coding RNA molecule (containing about 22 nucleotides) found in plants, animals and some viruses, that functions in RNA silencing and post-transcriptional regulation of gene expression. The miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. Under a standard nomenclature system, names are assigned to experimentally confirmed miRNAs. The prefix “miR” is followed by a dash and a number, the latter often indicating order of naming. “MIR” refers to the gene that encodes a corresponding miRNA. Different miRNAs with nearly identical sequences except for one or two nucleotides are annotated with an additional lower case letter.

The term miRNA mimics, as used herein, refers to small, double-stranded RNA molecules, such as siRNA, designed to mimic endogenous mature miRNA molecules when introduced into cells. In some figures, the miR-16 mimics are designated Mir-16 mimics

The term “inhibitory oligonucleotide” refers to any oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include isolated or synthetic shRNA or DNA, siRNA or DNA, antisense RNA or DNA, Chimeric Antisense DNA or RNA, miRNA and miRNA mimics, among others.

The term “connexin” as used herein means a large family of trans-membrane proteins that allow intercellular communication and the transfer of ions and small signaling molecules and assemble to form gap junctions. Connexins are four-pass transmembrane proteins with both C and N cytoplasmic termini, a cytoplasmic loop (CL) and two extra-cellular loops, (EL-1) and (EL-2). Connexins are assembled in groups of six to form hemichannels, or connexons, and two hemichannels, one on each cell, then combine to form a gap junction between the two cells. The connexin gene family is diverse, with twenty-one identified members in the sequenced human genome, and twenty in the mouse (nineteen of which are orthologous pairs). They usually weigh between 26 and 60 kiloDaltons (kDa), and have an average length of 380 amino acids. The various connexins have been observed to combine into both homomeric gap junctions (both connexins the same) and heteromeric gap junctions (two different connexins), each of which may exhibit different functional properties including pore conductance, size selectivity, charge selectivity, voltage gating, and chemical gating. The term Connexin is abbreviated as Cx and the gene encoding for it CX. In recent literature, connexins are commonly named according to their molecular weights, e.g. Cx26 is the connexin protein of 26 kDa, using the weight of the human protein for the numbering of orthologous proteins in other species.

The term “gap junction” as used herein means a specialized intercellular connection between a multitude of animal cell-types. They directly connect the cytoplasm of two cells, which allows various molecules, ions and electrical impulses to directly pass through a regulated gate between cells.

The term syncytial refers to a syncytial tissue that is made up of cells interconnected by specialized membrane with gap junctions, which are synchronized electrically in an action potential. Syncytial cells include a cardiac myocyte, a smooth muscle cell, an epithelial cell, a connective tissue cell, or a syncytial cancer cell.

The term “delivering” or “delivered” as used herein means introducing a molecule into an inside of a cell membrane.

The term “donor cell” as used herein means a cell that has been loaded with a molecule to be delivered to a different cell called a target cell.

The term “target cell” as used herein means a cell selectively affected by a particular agent, such as a donor cell or content carried by the donor cell.

The term “human mesenchymal stem cell,” abbreviated hMSC) as used herein, means a human multipotent stromal cell that can differentiate into a variety of cell types, including: human osteoblasts (bone cells), human chondrocytes (cartilage cells), human myocytes (muscle cells) and human adipocytes (fat cells).

The terms “individual,” “subject,” “host,” and “patient,” are used interchangeably herein and refer to any mammalian subject for whom diagnosis, treatment, or therapy is desired, particularly humans.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### 2. Overview

According to our own earlier work, a method of delivering an oligonucleotide or a plasmid expressing an oligonucleotide into a target cell includes introducing an oligonucleotide into a donor cell, and contacting the target cell with the donor cell under conditions permitting the donor cell to form a gap junction with the target cell, whereby the oligonucleotide or a product of the oligonucleotide is delivered into the target cell from the donor cell.

According to the some new embodiments, the donor cell is a human mesenchymal stem cell (hMSC), the inhibitory oligonucleotide is an inhibitory oligonucleotide that can pass through gap junctions, and the target cell is a cell of syncytial cancer tissue. The syncytial cancer tissue is also called a syncytial cancer tumor, herein. The target cell is also called a syncytial cancer cell or a tumor cell, herein. The method is shown to be effective in retarding the growth of syncytial cancer tumors. This is surprising because past studies have shown that hMSC enhance cancer tumor growth. This is also surprising because it was not previously known that the inhibitory oligonucleotide can be loaded into the donor cell without damage to the donor cell, which damage could render the donor cell unable to survive long enough to make an effective delivery of the inhibitory oligonucleotide.

In various embodiments, the oligonucleotide is an inhibitory oligonucleotide that can traverse the gap junction. The oligonucleotide may be DNA, such as a plasmid, that codes for a siRNA, antisense RNA, miRNA or miRNA mimic The oligonucleotide may be an antisense oligonucleotide or a cDNA that produces an inhibitory oligonucleotide that can traverse the gap junction. The oligonucleotide may be a mRNA or a cDNA that produces a siRNA or miRNA, or a siRNA mimic of miRNA, that can traverse the gap junction. In some embodiments, the oligonucleotide is a plasmid that encodes for siRNA. The oligonucleotide may comprise 12-28 members or more.

The gap junction channels may be composed of one or more of connexin 43 (Cx43), connexin 40 (Cx40), connexin 45 (Cx45), connexin 32 (Cx32) and connexin 37 (Cx37) among others present in syncytial cancer tissue. In some embodiments Cx32 is not used to pass siRNA.

The syncytial tumors treated in various embodiments include tumors of cervical cancer, colorectal cancer, melanoma, pancreatic cancer, and prostate cancer.

Our own earlier work provide a way to pass oligonucleotides (DNA and/or RNA fragments) through gap junction channels. This has been demonstrated in experiments where gap junction channels composed of connexin 43 (Cx43) were used in a HeLa cell line for cervical cancer.

The experiments determined that oligo complexes such as DNA or RNA sequences of defined length are able to pass through a gap junction channel. DNA or RNA, forms alpha helixes in solution with minor diameters of 0.9-1.0 nm. Oligonucleotides in the 12-24 nucleotide or nucleotide pair size range are of particular interest. In various experiments, unique sequences of DNA which could not be broken down into smaller fragments, called Morpholinos, were tagged with a fluorescent probe from GENE TOOLS, LLC™ of Philomath, Oregon which specializes in the manufacture of oligo sequences. Hek 293 parental cells were grown on 18×18 mm sterile coverslips that were placed within 35 mm culture dishes. Approximately 24 hours post seeding the culture medium on each was replaced with 2 ml of fresh complete medium (10% FBS, 1% P/S) to which a 24-mer morpholino (Gene Tools) and Endo-Porter (Gene Tools) were added. The morpholino final concentration was 1.25 uM, the Endo-Porter final concentration was ˜6 uM. The morpholino remained on the cells for maximal delivery, no washing. The control dish received complete medium with Endo-Porter only Coverslips were fixed at various time points with 3.7% formaldehyde. The coverslips were mounted with Vectashield (Vector Labs), images were captured on an Olympus Fluoview 1000 confocal microscope using a 63× oil objective. Fluorescence intensity profiles were made by using the Olympus line series analysis software tool.

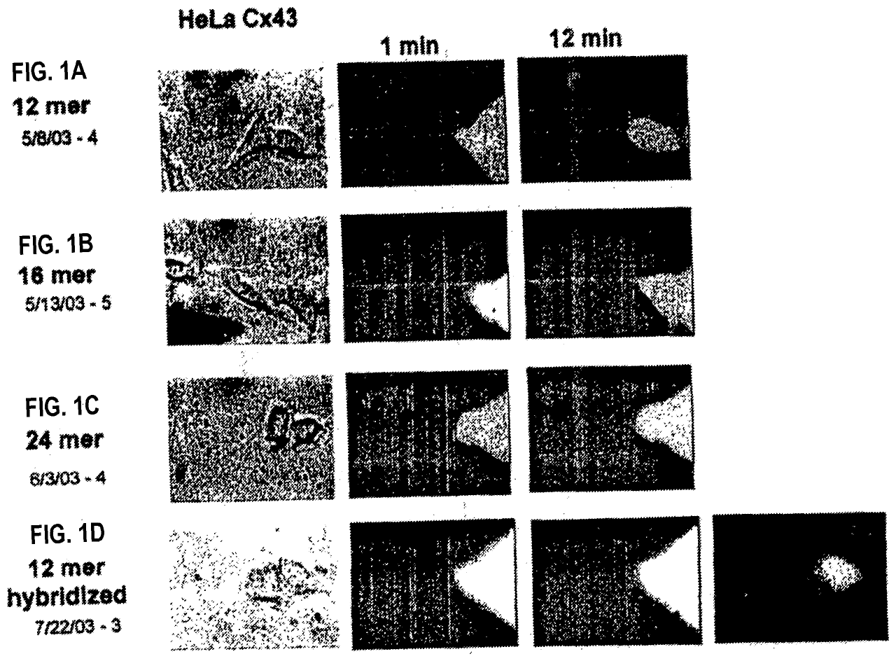

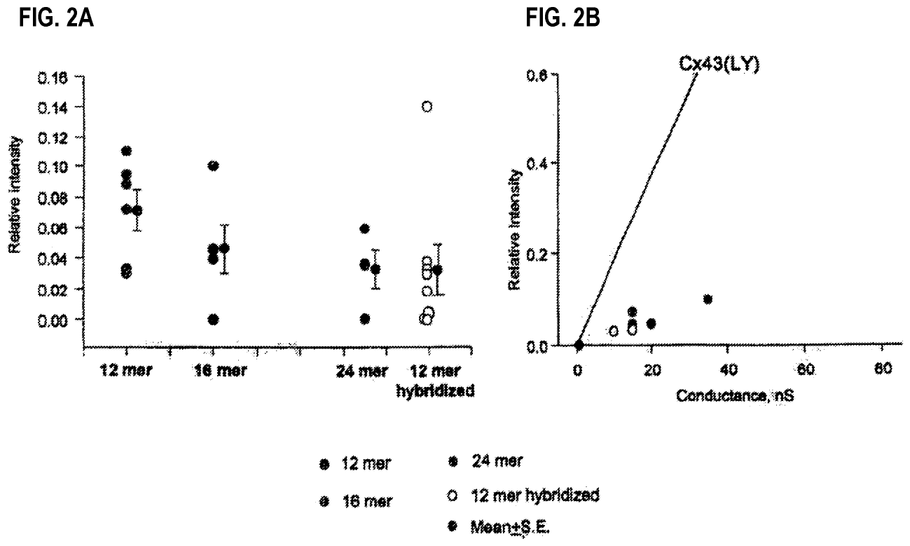

A first set of experiments were conducted with a 12 member oligonucleotide, a 16 member oligonucleotide and a 24 member oligonucleotide. The results demonstrated that all three single stranded forms pass through gap junction channels composed of Cx43 (FIG. 1A, FIG. 1B, and FIG. 1C). Further, two 12 member compliments were hybridized producing a double stranded form and its passage was measured (FIG. 1D). The double stranded version has only a small increase in its minor diameter. FIG. 2A shows a summary of the data where the X-axis is the length of the oligonucleotide. The hybridized 12 member oligonucleotide is plotted out of sequence on the X-axis. The Y-axis is the relative intensity of the fluorescent tag in the recipient cell (the cell on the left in all of the examples of FIG. 1A through FIG. 1D) 12 minutes after delivery of the oligonucleotide to the source cell. For each oligonucleotide, the individual experimentally derived values are shown along with the mean and standard deviation for each oligonucleotide. In a number of experiments junctional conductance and the transfer of fluorescently labeled oligonucleotide were monitored simultaneously. FIG. 2B is a graphic representation of junctional conductance on the X-axis versus relative intensity of the fluorescent tag on the Y-axis. For comparison the conductance-intensity relationship for Lucifer Yellow passage through Cx43 gap junction channels is shown (Valiunas et al., 2002) (2). In all cases the relative intensity, which represents the transfer rate from one cell to another, is 5-10 times less than the Lucifer Yellow fluorescence intensity in recipient cells. This lower transfer rate is consistent with the rod-like dimensions and molecular weight of the oligonucleotide, whose minor diameter is 1.0 nanometers (nm), being less mobile in solution than Lucifer Yellow.

These observations demonstrate that gap junction channels are a feasible delivery port for molecules such as silencing RNA (siRNA) or any other molecule of similar dimension. We have previously demonstrated that hMSCs make gap junctions with each other and target cells. We have also demonstrated previously that one can load plasmids into stem cells by electroporation. The present results demonstrate that any donor cell type which forms gap junctions with another target cell type (this includes hMSCs as potential donor or target cells) can be used as a vehicle to deliver RNA or DNA.

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

In the example embodiments, the oligonucleotide is an siRNA or miRNA or an siRNA mimic of miRNA (called “miRNA mimic” hereafter for convenience), such as one or more of miR-16, miR-34a, miR-16 mimic, miR-34a mimic, Cortactin siRNA, Gelsolin siRNA, Akt siRNA, α-Tubulin siRNA, GAPDH siRNA, Kras^{GAT} siRNA or DNA that codes for such siRNA, that interferes with tumor growth. These inhibitory oligonucleotide were originally chosen because of their known properties in affecting essential cell structures or processes or apoptosis (cell death). Of these, certain inhibitory oligonucleotide were identified as advantageous in the following experimental embodiments.

For example, in some embodiments, the miR-16 is Gene ID: 406950 - MIR16-1 microRNA 16-1 and its corresponding miR-16 mimic is CCAGUAUUAACUGUGCUGCUGA (hsa-mir-16-1, SEQ ID NO: 1). In other embodiments the mirR-34a is Gene ID: 407040 - MIR34A microRNA 34a and its corresponding miR-34a mimic is UGGCAGUGUCUUAGCUGGUUGU (hsa-mir-34a, SEQ ID NO: 2).

### 3. Experimental Embodiments

The following experiments demonstrate that certain inhibitory oligonucleotide: can retard syncytial cancer tumor growth in vitro; can be loaded into hMSC in vitro without preventing survival of the hMSC; can be passed between hMSC and syncytial cancer cells via gap junctions; can be propagated via gap junctions among cells within a tumor; and, when loaded into hMSC and contacted to such tumors, can effectively and therapeutically retard or reduce tumor growth in vivo. For these reasons, various embodiments are anticipated to provide an effective treatment for syncytial cancer tumors.

In various in vitro direct transfection experiments, described below, hMSCs or other cells are directly transfected with an siRNA to produce loaded cells. Unless otherwise specified, these experiments were performed in a petri dish containing identical cell culture medium under sterile conditions well known in the art. Transfection occurred over a 24 hour transfection period with a solution of the inhibitory oligonucleotide, typically as a 100 nanoMolar (nM) solution, and with a transfection agent (lipofectamine), with cell densities of about 20% to about 30%. At the end of the 24 hour period, the cells were rinsed with culture media and the hMSC or other cell population was introduced to an indirect transfection experiment, described below, or recorded over time, using any cell count method, including absorbance or fluorescence intensity of any labels, to yield cell proliferation over time.

In various in vitro indirect transfection experiments, the loaded or unloaded hMSCs or other cells were co-cultured with a target cell population. When loaded, the hMSCs had been directly transfected as described above. Unless otherwise specified, these experiments were performed by plating the loaded or unloaded hMSCs onto a petri dish with target cells at about 20% to 30% confluence. The co-plated cells were then rinsed with culture media. Then the target cell population was recorded over time, using any cell count method, including absorbance or fluorescence intensity of any labels, to yield target cell proliferation over time.

### 3.1 Treatment In Vivo with hMSC can Enhance Tumor Growth

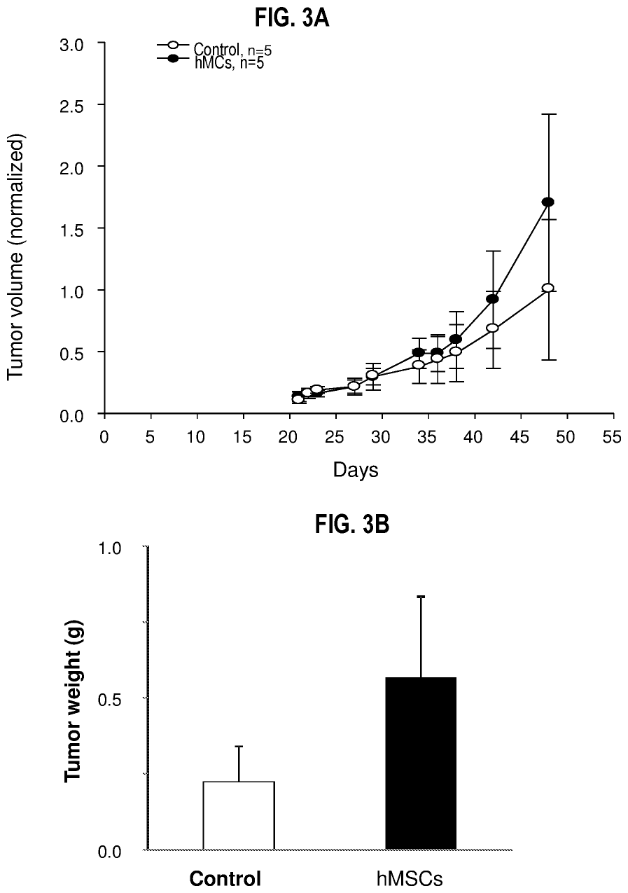

FIG. 3A and FIG. 3B are graphs that illustrate example increase in tumor growth rate when contacted in vivo with human mesenchymal stem cells (hMSCs). FIG. 3A is a graph that illustrates tumor volume growth for tumor cells in mice (in vivo) grown alone (control) and in the presence of hMSCs that are not loaded with certain inhibitory oligonucleotide. The horizontal axis indicates elapsed time in days and the vertical axis indicates normalized tumor volume. The tumor is a PC-3 cell line of human prostate cancer used in prostate cancer research. These cells are useful in investigating the biochemical changes in advanced prostatic cancer cells and in assessing their response to treatments. Moreover, they can be used to create subcutaneous tumors in mice in order to investigate an in vivo model of the tumor environment in the context of the organism.

The open circles show the PC-3 tumor volume for the control at various times between 20 days and 50 days after introduction into nude mice averaged over five different mice. The solid circles show the PC-3 tumor volume for the tumors grown with hMSCs at various times between 20 days and 50 days after introduction into nude mice averaged over another five different mice. In this experiment PC-3 cells were injected with an equal number of hMSCs. The graph is normalized to the control value at 48 days. The vast majority of the injected hMSCs are unlikely to stay at the tumor site for more than about 3 to 4 days. The standard deviation of the results are indicated by vertical bars. The tumor grown with hMSCs consistently measured greater volume than the control.

At the end of the experiment, the animals were sacrificed and the tumors weighed. FIG. 3B is a bar graph that illustrates tumor weight for tumor cells grown alone (control) and in the presence of hMSCs that are not loaded with certain inhibitory oligonucleotide. The horizontal axis indicates the group and the vertical axis indicates weight in grams. Tumor weight in FIG. 3B is estimated from tumor volume that was obtained by imaging of the green fluorescent protein (GFP) that the tumor cells expressed. The open bar indicates the average weight of the control group and the solid bar the avenge weight of the group grown with hMSCs, the vertical lines indicate the standard deviation. There is a significant increase in tumor growth associated with treatment by hMSCs that are not loaded with inhibitory oligonucleotide.

### 3.2 Certain siRNA can Retard Tumor Growth by Direct Exposure in vitro

Research was performed to discover candidate inhibitory oligonucleotide for experimental testing and then experiments were conducted in vitro to discover the most effective inhibitory oligonucleotide suitable for introduction into corresponding tumors in vivo via hMSCs. Candidate inhibitory oligonucleotide include siRNA directed against Gelsolin, GAPDH, c Tubulin, Cortactin, and Akt, which play roles in cellular structure and primary functions. If such structural proteins or primary functions were limited in target cells, it was anticipated that the affected cells could not function properly and would grow more slowly. While the above protein targets were identified in the illustrated embodiments, in various other embodiments, other cellular proteins are targeted by inhibitory oligonucleotide.

As is known in the art, Gelsolin is an important actin regulator, and plays a role in podosome formation (along with Arp3, Cortactin, and Rho GTPases) which effect cell motility and which are exhibited in many different specialized cells such as invasive cancer cells. Gelsolin also inhibits apoptosis (cell death) by stabilizing the mitochondria. Glyceraldehyde 3-phosphate dehydrogenase (abbreviated as GAPDH) is an enzyme of ˜37 kDa in size that catalyzes the sixth step of glycolysis and thus serves to break down glucose for energy and carbon molecules. Both α- and β-tubulins polymerize into microtubules, a major component of the eukaryotic cytoskeleton. Microtubules function in many essential cellular processes, including mitosis. Cortactin is present in all cell types; it is a monomeric protein located in the cytoplasm of cells that can be activated by external stimuli to promote polymerization and rearrangement of the actin cytoskeleton, especially the actin cortex around the cellular periphery. Akt, also known as Protein kinase B (PKB), is a serine/threonine-specific protein kinase that plays a key role in multiple cellular processes such as glucose metabolism, apoptosis, cell proliferation, transcription and cell migration.

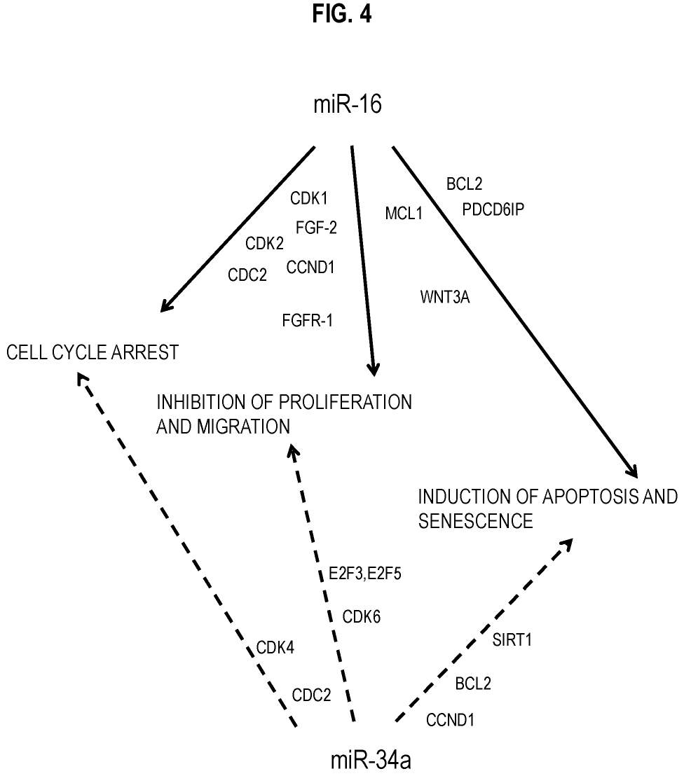

Other candidate inhibitory oligonucleotide were selected among microRNA known to affect various important cell process pathways. FIG. 4 is a diagram that illustrates microRNA that interfere with various cell process pathways; and, thus represent potential agents for retarding tumor growth, according to an embodiment. For example, miR-16 and miR-34a are known to interfere with the translation of various proteins. While the above miRNA and their siRNA mimics were identified in the illustrated embodiments, in various other embodiments, other miRNA and their siRNA mimics are used as inhibitory oligonucleotide. As shown in FIG. 4, miR-16 interferes with the translation of proteins CDK1, CDK2 and CDC2; and, miR-34a interferes with the translation of proteins CDC2 and CDK4. These proteins play roles in a cell's growth and division cycle, thus such interference can potentially lead to cell cycle arrest. Similarly, miR-16 interferes with the translation of proteins FGF-2, CCND1 and FGFR-1; and, miR-34a interferes with the translation of proteins CDK5, E2F3 and E2F5. These proteins play roles in a cell's proliferation (increase in numbers) and migration, thus such interference can potentially lead to inhibition of such proliferation and migration. As also depicted in FIG. 4, miR-16 interferes with the translation of proteins BCL2, PDC6IP, MCL1 and WNT3A; and, miR-34a interferes with the translation of proteins CCND1, BCL2 and SIRT1. These proteins play roles in delaying apoptosis (cell death) and senescence (cell aging), thus such interference can potentially lead to inducing senescence or apoptosis, and thereby inhibiting tumor growth.

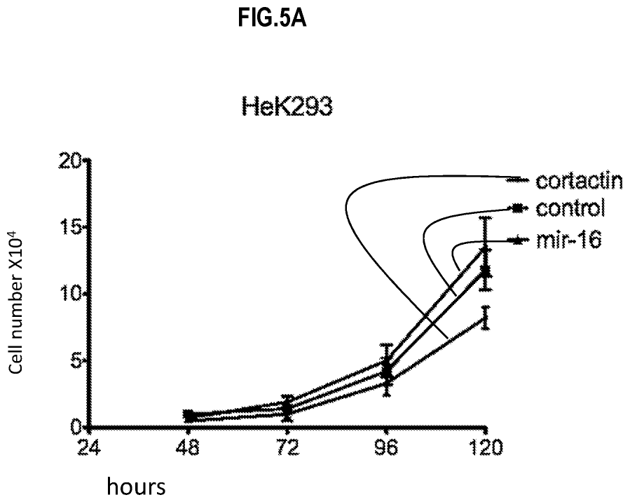

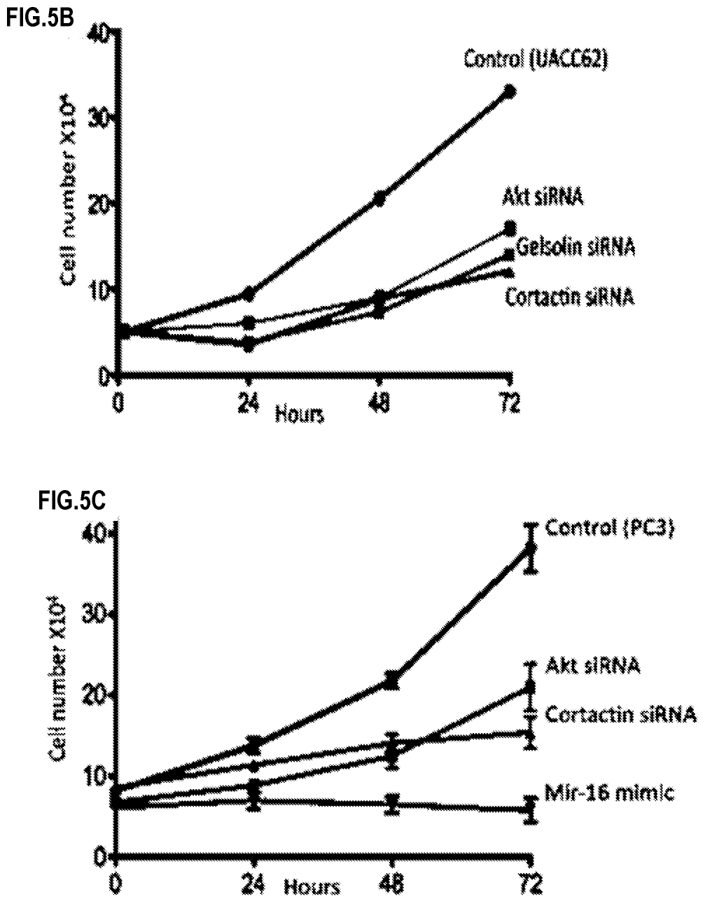

FIG. 5A through FIG. 5C are plots that illustrate relative effects on tumor growth of various syncytial cancers or cells by potential agents for retarding tumor growth transfected directly in vitro; and thus indicate candidate agents for introduction via hMSCs, according to an embodiment. FIG. 5A depicts the effects of two types of inhibitory oligonucleotide on growth for normal Human Embryonic Kidney 293 cells, also called HeK293 herein. HeK293 cells are a specific non-cancer cell line originally derived from human embryonic kidney cells grown in tissue culture. HEK293 cells are very easy to grow and transfect very readily and have been widely used in cell biology research. Direct transfection experimental protocols were used with a 24 hours transfection period. The horizontal axis indicates hours after start of the experiment. The vertical axis indicates the number of cells in units of ten thousand cells. The vertical lines indicate standard deviation across n=6 experiments to which the label applies. The trace labeled “control” is the growth curve for normal HeK293. The trace labeled “cortactin” refers to cells grown after exposure at 48 hours to siRNA that interferes with Cortactin, i.e, Cortactin siRNA. The trace labeled “mir-16” refers to cells grown after exposure at 48 hours to siRNA that mimics miR-16, i.e., miR-16 mimic Clearly, Cortactin siRNA is effective at reducing cell growth in HeK293, indicating Cortactin siRNA may be effective at controlling growth when transfected into other cell types, such as cancer cells. As will be shown below, when transfected into cancer cells miR-16 mimics are more effective than they are for the normal HeK cells shown in FIG. 5A.

FIG. 5B depicts the effects of three types of inhibitory oligonucleotide on growth for human melanoma represented by the UACC-62 cell line, also called UACC62 cells herein. Direct transfection experimental protocols were used, with n=6 experiments for each trace. The horizontal axis indicates hours after start of the experiment. The vertical axis indicates the number of cells in units of ten thousand cells. The trace labeled “control (UACC62)” is the growth curve for UACC-62 cells without loading by inhibitory oligonucleotide. The traces labeled “Cortactin siRNA, Gelsolin siRNA, Akt siRNA” refer respectively to cells grown after exposure starting at 0 hours to siRNA that interferes with Cortactin, Gelsolin and Akt. Clearly, Cortactin siRNA, Gelsolin siRNA, Akt siRN all reduce the rate of proliferation, indicating all may be effective at controlling growth of melanoma tumors.

FIG. 5C depicts the effects of three types of inhibitory oligonucleotide on growth for human prostate cancer represented by the PC-3 cell line, also called PC3 cells herein. Direct transfection experimental protocols were used, with n=6 experiments for each trace. The horizontal axis indicates hours after start of the experiment. The vertical axis indicates the number of cells in units of ten thousand cells. The vertical lines indicate standard deviation among the 6 different experiments of the labeled type. The trace labeled “control (PC3)” is the growth curve for PC-3 cells without loading by inhibitory oligonucleotide. The traces labeled “Cortactin siRNA, Akt siRNA and Mir-16 mimic” refer respectively to cells grown after exposure at 0 hours to siRNA that interferes with Cortactin and Akt and that mimics miR-16. Clearly, both Cortactin siRNA and Akt siRN reduce the rate of proliferation, indicating both may be effective at controlling growth of prostate tumors. It is also clear that the miR-16 mimic is even more effective against prostate cancer; and, this suggests that miR-16 mimic may be more effective than either Akt siRNA or Cortactin siRNA against other cancer types, including melanoma.

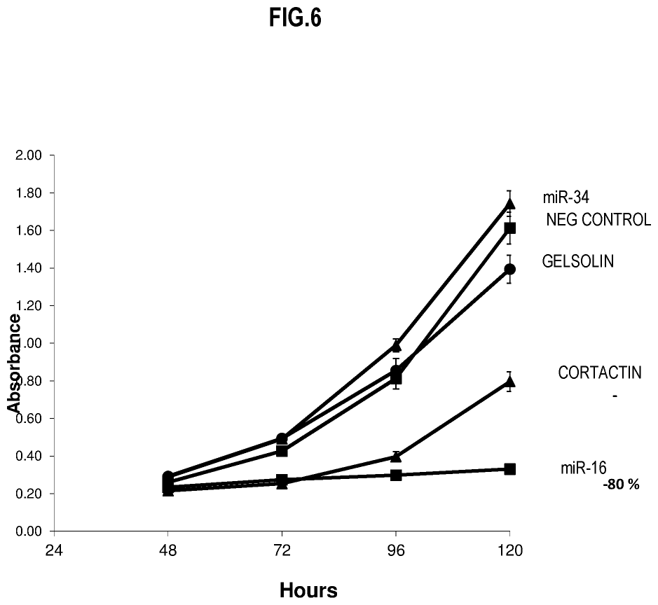

FIG. 6, is a plot that illustrates relative effects on melanoma tumor growth by potential agents, including miR-16, for retarding tumor growth transfected directly in vitro; and thus indicate candidate agents for introduction via hMSCs, according to an embodiment. Direct transfection experimental protocols were used, with n=6 experiments for each trace. The horizontal axis indicates hours after start of the experiment. The vertical axis indicates the absorbance of an optical beam, which is related to the number of cells. The trace labeled “NEG CONTROL” indicates a growth curve for UACC-62 cells without loading by inhibitory oligonucleotide. The traces labeled “CORTACTIN, GELSOLIN” refer respectively to cells grown after direct exposure to siRNA that interferes with Cortactin and Gelsolin. The traces labeled “miR-16, miR-34” refer respectively to cells grown after direct exposure to siRNA that mimics miR-16 and siRNA that mimics miR-34a. The value at each point of each trace indicates an average and the vertical lines at each point indicates the standard deviation over 6 experiments of each labeled type. Gelsolin siRNA and miR-34a mimics do not appear to usefully reduce proliferation of this melanoma cell line. This corroborates the result in FIG. 5B that Cortactin siRNA appears to be effective, with an estimate rate reduction of about 50%. This further demonstrates that siRNA that mimics miR-16 is indeed more effective than Cortactin siRNA in reducing the rate of melanoma proliferation, at least for this cell line, with an better estimated rate reduction of about 80%.

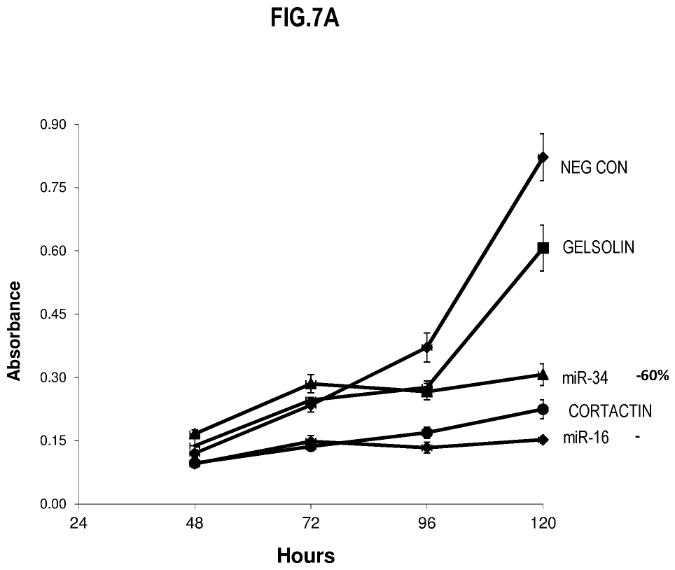

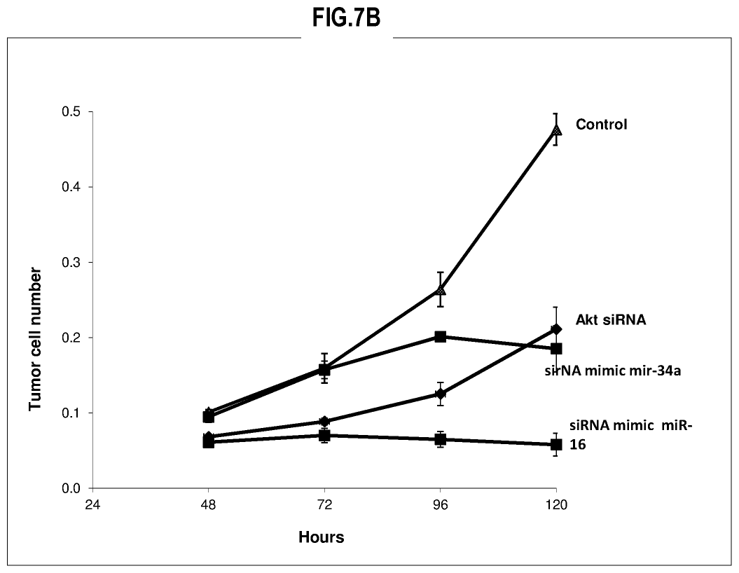

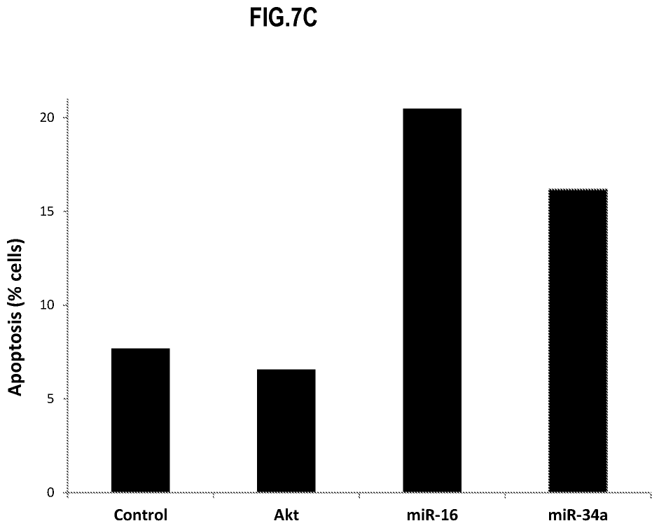

FIG. 7A through FIG. 7C are plots that illustrate relative effects on prostate tumor growth by potential agents, including miR-16 or mimics thereof, for retarding tumor growth transfected directly in vitro; and thus indicate candidate agents for introduction via hMSCs, according to an embodiment. Direct transfection experimental protocols were used, with n=6 experiments for each trace. In FIG. 7A, the horizontal axis indicates hours after start of the experiment. The vertical axis indicates the absorbance of an optical beam, which is related to the number of cells. The trace labeled “NEG CON” indicates a growth curve for PC-3 cells without loading by inhibitory oligonucleotide. The traces labeled “CORTACTIN, GELSOLIN” refer respectively to cells grown after direct exposure to siRNA that interferes with Cortactin and Gelsolin. The traces labeled “miR-16, miR-34” refer respectively to cells grown after direct exposure to siRNA that mimics miR-34 and siRNA that mimics miR-16. The value at each point of each trace indicates an average and the vertical lines at each point indicates the standard deviation over 6 experiments of each labeled type. All siRNA have some effect on reducing cell proliferation, with Gelsolin siRNA having the smallest effect. Cortactin siRNA appears to be effective, with an estimate rate reduction of about 60%, with miR-34 mimics slightly less effective and miR-16 mimics even more effective than Cortactin siRNA.

In FIG. 7B the horizontal axis is the same as in FIG. 7A; but, the vertical axis indicates the number of cells in the tumor in relative units. The trace labeled “CONTROL” indicates a growth curve for PC-3 cells without loading by inhibitory oligonucleotide. The trace labeled “Akt siRNA” refers to cells grown after direct exposure to siRNA that interferes with Akt. The traces labeled “SiRNA mimic mir-16, SiRNA mimic miR-34” refer respectively to cells grown after direct exposure to siRNA that mimics miR-34 and siRNA that mimics miR-16. The value at each point of each trace indicates an average and the vertical lines at each point indicates the standard deviation over several experiments of each labeled type. All siRNA have some effect on reducing cell proliferation, with miR-16 mimics the most effective.

FIG. 7C is a bar graph that illustrates percentage of cells that experience apoptosis for PC-3 tumor cells grown alone (control) and after transfection of the inhibitory oligonucleotide of FIG. 7B. The horizontal axis indicates the group, where the labels Akt, miR-16 and miR-34a indicate respectively the groups transfected with Akt siRNA, siRNA mimic for miR-16 and siRNA mimic for miR-34a. The vertical axis indicates apoptosis in percentage of cells as determined via Terminal deoxynucleotidyl transferase dUTP nick end labeling (TUNEL) assay. A TUNEL assay is a method for detecting DNA fragmentation by labeling the terminal end of nucleic acids; and, is a common method for detecting DNA fragmentation that results from apoptotic signaling cascades. There is a significant increase in apoptosis associated with direct transfection of SiRNA mimics for miR-16 and miR-34a, with mimics for miR-16 being the most effective. The sample was subjected to the TUNEL assay at the end of the experiment at 120 hours.

In all the above experiments, mimics of miR-16 proved the most effective at reducing cell proliferation; and, so was used for most of the remaining experiments and the first in vivo experiments presented in a later section.

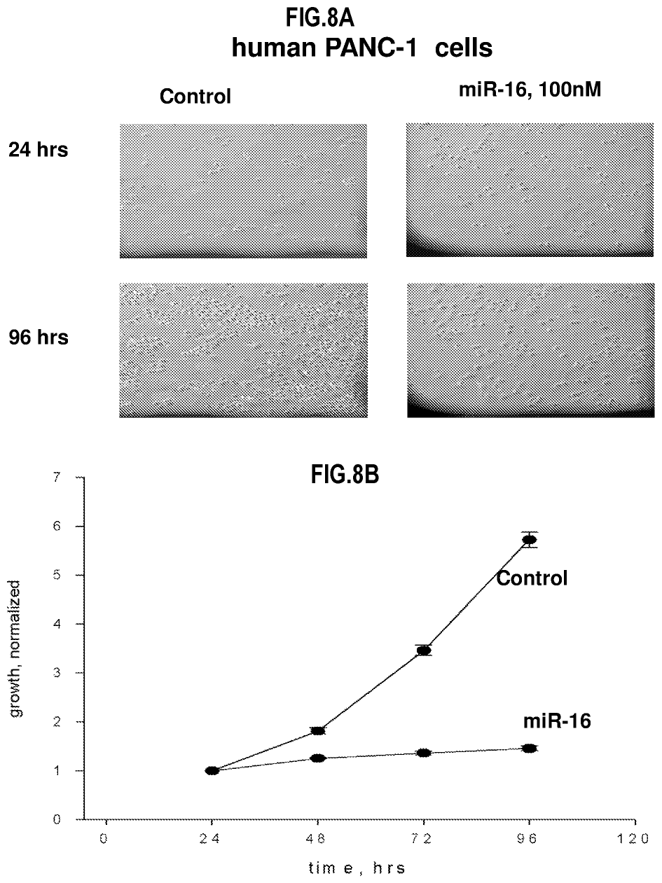

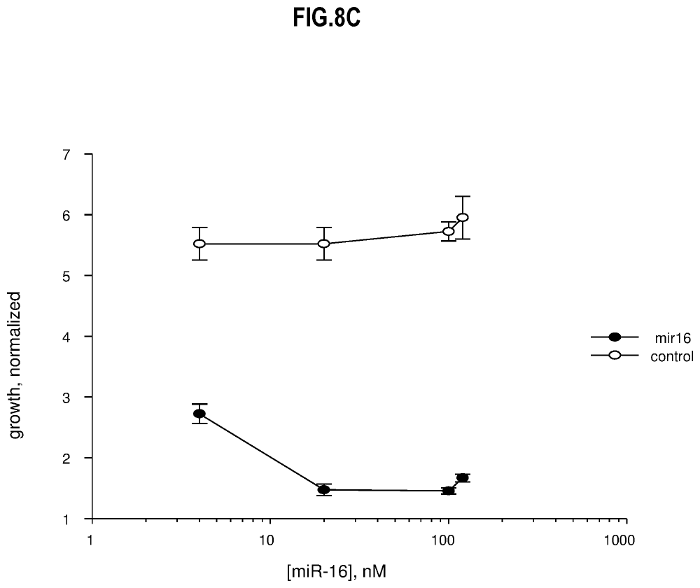

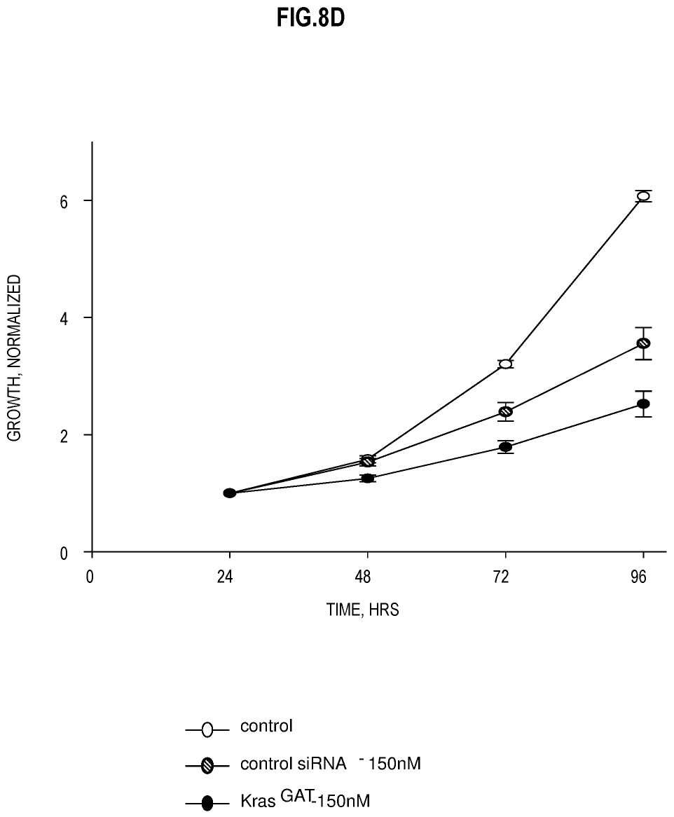

FIG. 8A through FIG. 8D are images and plots that illustrate relative effects on pancreatic tumor growth by potential agents, including miR-16 and Kras^{GAT} SiRNA, for retarding tumor growth transfected directly in vitro; and thus indicate candidate agents for introduction via hMSCs, according to an embodiment. Non endocrine pancreatic cancer is modeled using the PANC-1 cell line for human pancreatic epithelioid carcinoma. Direct transfection experimental protocols were used, with n=4 experiments for each trace. FIG. 8A depicts four images. The two images on the left depict micrographs of cells of human PANC-1 cells in culture at 24 hours and 96 hours after start of the experiment. The two images on the right depict micrographs of cells of human PANC-1 cells in culture at 24 hours and 96 hours after start of the experiment subjected to direct transfection in a solution of 100 nano Molar (nM) siRNA that mimics miR-16. As can be seen by comparing the two images at 96 hours, there is a clear reduction in proliferation of PANC-1 cells in the culture exposed to miR-16 mimic.

This result is quantified in FIG. 8B which depicts PANC-1 cell population growth (proliferation) against time. The horizontal axis indicate time in hours, and the vertical axis indicates population normalized by the control population at 24 hours. The control trace indicates the population size for PANC-1 cells not exposed to an inhibitory oligonucleotide. The miR-16 race indicates the population size for PANC-1 cells exposed to the 100 nM solution of siRNA that mimics miR-16. The population exposed to miR-16 mimics of about 1.3 times the 24 hour population is reduced by almost 80% at 96 hours from the control population of about 5.7 times the 24 hour population. This same reduction can be achieved at exposures to even lower concentrations of miR-16 mimics

FIG. 8C is a plot that illustrates example PANC-1 cell line population reductions at 96 hours for different concentrations of miR-16 mimics; and, thus plots a dose response to exposure to miR-16. The logarithmic horizontal axis indicates concentration of miR-16 mimics in units of nanoMolar (nM); and, the vertical axis is the same as in FIG. 8B. The control trace is plotted with open circles and the trace for exposure to the miR-16 mimic solution is plotted with solid circles. The traces in FIG. 8B were plotted for a miR-16 mimic concentration of 100 nM. At 100 nM in FIG. 8C, one can see the same result: the population exposed to miR-16 mimics is at about 1.3 almost 80% reduced from the control population of about 5.7. The difference is about the same at slightly more than 100 nM and at about 20 nM. Even at a concentration of only 4 nM, the population of the cells exposed to miR-16 mimics is significantly reduced, at about 2.7 compared to the control population at about 5.5, for a reduction of about 50%. There seems to be no advantage to using the higher concertation 100 nM solution compared to 20 nM solution.

FIG. 8D is a plot that illustrates effects of other siRNA on the PANC-1 cell line. KRAS and BRAF are oncogenes involved in the epidermal growth factor receptor (EGFR) signaling pathway that controls cell proliferation, differentiation and apoptosis. Mutations in the KRAS and BRAF oncogenes are frequently found in human syncytial cancers, such as colorectal cancers and non-small cell lung cancers. However, non-small cell lung cancers have reduced connexin expression so they are not ideal candidates for the proposed treatments involving gap junction delivery of siRNA. In some cancers a gene mutation in codon 12 is observed and called the GAT mutation. Thus siRNA (called Kras^{GAT} siRNA herein) interfering with the translation of the protein (called Kras^{GAT} herein) coded for by this mutation may have beneficial effects in fighting the spread of such cancers. The horizontal axis indicates time in hours, and the vertical axis indicates population normalized by the control population at 24 hours. The control trace uses open circles and indicates the population size for PANC-1 cells not exposed to an inhibitory oligonucleotide. The Kras^{GAT} trace uses solid circles and indicates the population size for PANC-1 cells exposed to a 150 nM solution of Kras^{GAT} siRNA. The control siRNA trace uses circles filled with diagonal hatches and indicates the population size for PANC-1 cells exposed to a 150 nM solution of siRNA that does not interfere with any major pathway or structure. The control siRNA is of the same length as the Kras^{GAT} siRNA, but the control siRNA does not code for any gene and is called a nonsense siRNA. Transfection started at zero hours for both populations. This plot show that there is a moderate effect by Kras^{GAT} siRNA that is in excess of the nonsense siRNA.

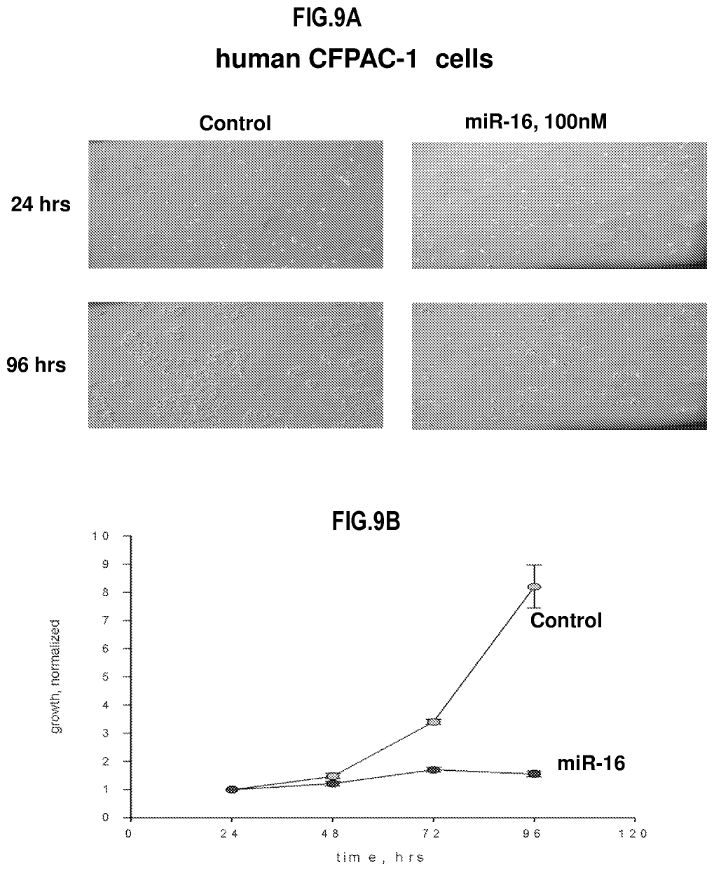

FIG. 9A is an image and FIG. 9B is a plot that both illustrate relative effects on pancreatic tumor growth of a different cell line by potential agents, including miR-16 mimic, for retarding tumor growth transfected directly in vitro; and thus indicate candidate agents for introduction via hMSCs, according to an embodiment. Direct transfection experimental protocols were used, with n=6 experiments for each trace. The cell line, designated CFPAC-1, is a human pancreatic adenocarcinoma cell line from a patient with cystic fibrosis. FIG. 9A depicts four images. The two images on the left depict micrographs of cells of human CFPAC-1 cells in culture at 24 hours and 96 hours after start of transfection. The two images on the right depict micrographs of cells of human CFPAC-1 cells in culture at 24 hours and 96 hours after start of direct transfection in a solution of 100 nM siRNA that mimics miR-16. As can be seen by comparing the two images at 96 hours, there is a clear reduction in proliferation of CFPAC-1 cells in the culture exposed to miR-16 mimics

Similar to FIG. 8A and FIG. 8B, the result of FIG. 9A is quantified in FIG. 9B which depicts CFPAC-1 cell population growth (proliferation) against time. The horizontal axis indicates time in hours, and the vertical axis indicates population normalized by the control population at 24 hours. The control trace indicates the population size for CFPAC-1 cells not exposed to an inhibitory oligonucleotide. The miR-16 trace indicates the population size for CFPAC-1 cells exposed to the 100 nM solution of siRNA that mimics miR-16. The population exposed to miR-16 mimics of about 1.5 times the 24 hour population is reduced by over 80% at 96 hours from the control population of about 8.3 times the 24 hour population.

In these experiments as well, siRNA mimics of miR-16 proved the most effective at reducing cell proliferation.

### 3.3 hMSC Cytoplasm can be Loaded In Vitro with siRNA that Retard Tumor Growth

For gap junctions to be effective in transfecting an agent, such as an inhibitory oligonucleotide, from a donor cell to a target cell, the agent should be plentiful in the cytoplasm of the donor cell and thus frequently in the vicinity of the gap junctions. During some transfection processes, the agent is loaded into the cell from the surrounding fluid via endocytosis. Endocytosis is a form of active transport in which a cell transports molecules (such as nucleic acids and proteins) into the cell by engulfing them, forming vesicles. This is an energy-using process. Endocytosis and its counterpart, exocytosis, are used by all cells because most chemical substances important to them are large polar molecules that cannot pass through the hydrophobic plasma or cell membrane by passive means. For agents loaded by endocytosis to be available for transfection through a gap junction, the vesicle walls should degrade and release the agent into the cytoplasm. The next experiments demonstrate effective movement of the siRNA into the cytoplasm of an hMSC.

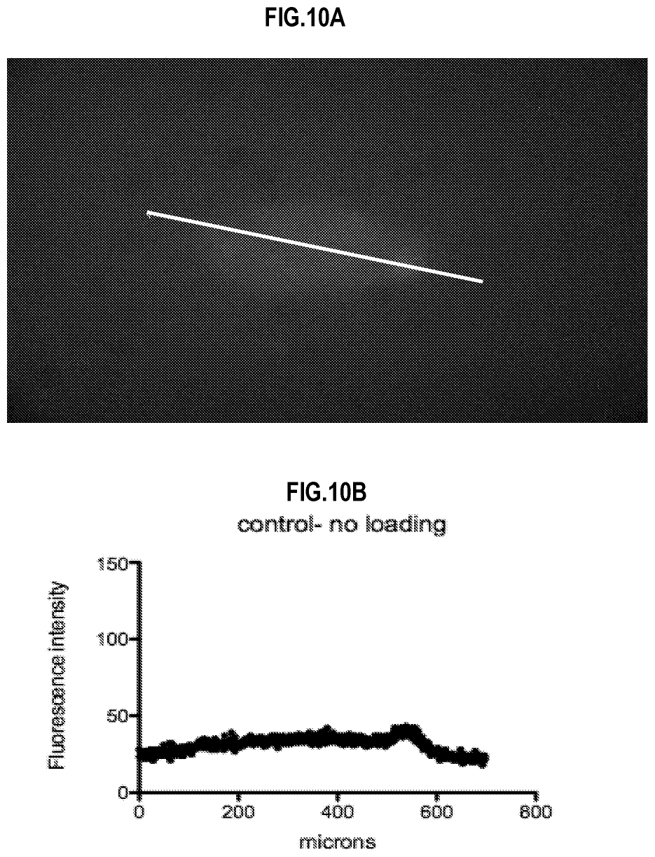

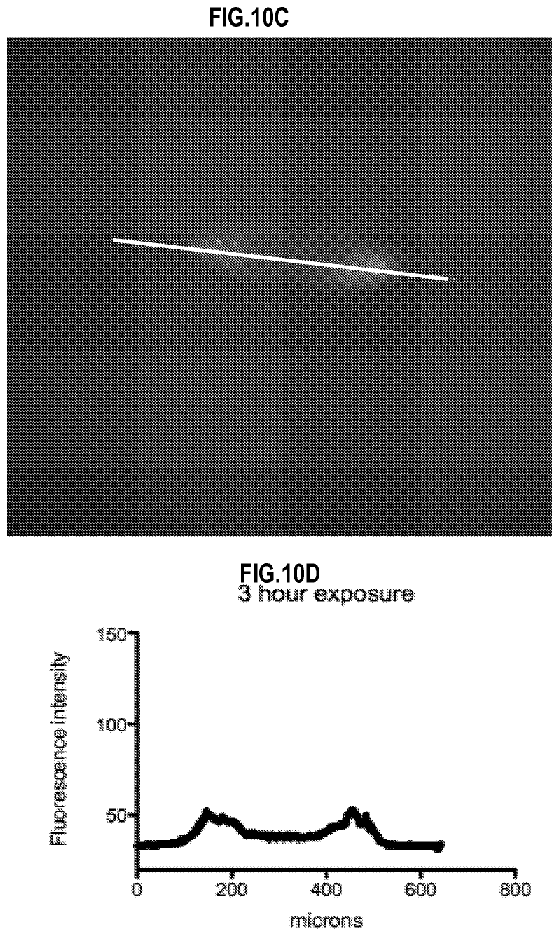

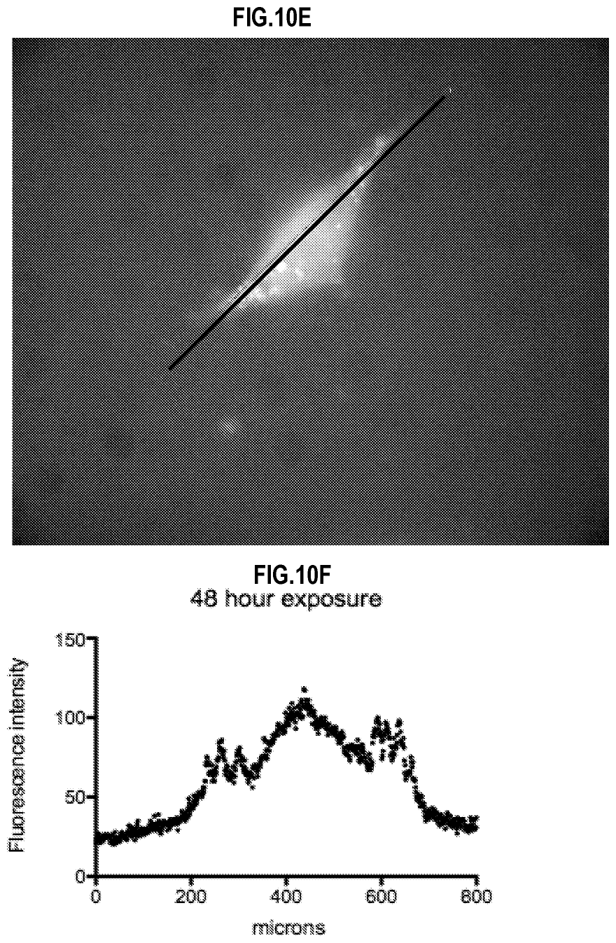

FIG. 10A through FIG. 10F are images and plots that illustrate loading of potential agents for retarding tumor growth transfected directly in vitro into hMSCs, according to an embodiment. In these embodiments, siRNA labeled with a fluorophore are loaded into a hMSC cell in culture. The labeled siRNA have a long dimension of about 500 microns (1 micron=1 micrometer, μm=10⁻⁶ meters. Hek 293 parental cells were grown on 18×18 mm sterile coverslips that were placed within 35 mm culture dishes. Approximately 24 hours post seeding the culture medium on each was replaced with 2 ml of fresh complete medium (10% FBS, 1% P/S) to which a 24 mer morpholino (Gene Tools) and Endo-Porter (Gene Tools) were added. The morpholino final concentration was 1.25 uM, the Endo-Porter final concentration was ˜6 uM. The morpholino remained on the cells for maximal delivery, no washing. The control dish received complete medium with Endo-Porter only Coverslips were fixed at various time points with 3.7% formaldehyde. The coverslips were mounted with Vectashield (Vector Labs), images were captured on an Olympus Fluoview 1000 confocal microscope using a 63× oil objective. Fluorescence intensity profiles were made by using the Olympus line series analysis software tool.

To distinguish the fluorescence by the loaded siRNA from the naturally occurring background fluorescence in an hMSC, the fluorescence is first measured for a control hMSC that has not been subjected to loading. FIG. 10A is an image that illustrates an example micrograph of fluorescence intensity from the control hMSC. There is a background fluorescence that is somewhat brighter in the oval shaped hMSC of the image. A profile of fluorescence intensity along an approximately 700 micron long white line segment in FIG. 10A is plotted in FIG. 10B. The horizontal axis indicates distance along the white line segment. The hMSC cell boundary is at approximately 100 microns and 600 microns, a length of about 500 microns. The vertical axis indicates fluorescence intensity in arbitrary units. There is a larger than average gradient in fluorescent intensity near the right side boundary of the hMSC.

FIG. 10C is an image that illustrates an example micrograph of fluorescence intensity from a different hMSC after 3 hours of exposure to loading with the fluorophore tagged siRNA. There is a fluorescence that is clearly brighter than the background at left and right edges of the oval shaped hMSC of the image. At both edges vesicles a few pixels across are apparent in which the fluorescence is very bright. A profile of fluorescence intensity along an approximately 700 micron long white line segment in FIG. 10C is plotted in FIG. 10D. The horizontal axis indicates distance along the white line segment. The hMSC cell boundary is at approximately 100 microns and 500 microns, a length of about 400 microns. The vertical axis indicates fluorescence intensity in arbitrary units There are two distinct peaks in fluorescent intensity near the left side and right side boundaries of the hMSC. The fluorescence still seems to be confined to vesicles near the cell boundary and not bright throughout the cytoplasm.

The story changes by 48 hours of exposure. FIG. 10E is an image that illustrates an example micrograph of fluorescence intensity from a different hMSC after 48 hours of exposure to loading with the fluorophore tagged siRNA. There is a background fluorescence but the triangular hMSC is clearly much brighter than the background. Vesicles a few pixels across are still apparent in which the fluorescence is extra bright. However, the fluorescence is bright throughout the cytoplasm. A profile of fluorescence intensity along an approximately 800 micron long line segment in FIG. 10E, which is black in this case to make it visible over the bright cytoplasm, is plotted in FIG. 10F. The horizontal axis indicates distance along the black line segment. The hMSC cell boundary is at approximately 200 microns and 700 microns, a length of about 500 microns. The vertical axis indicates fluorescence intensity in arbitrary units. There are two distinct peaks in fluorescent intensity near the left side and right side boundaries of the hMSC, but a broader and stronger peak in the middle of the cell. The fluorescence is bright throughout the hMSC. This is a favorable distribution of siRNA for transfection through gap junctions.

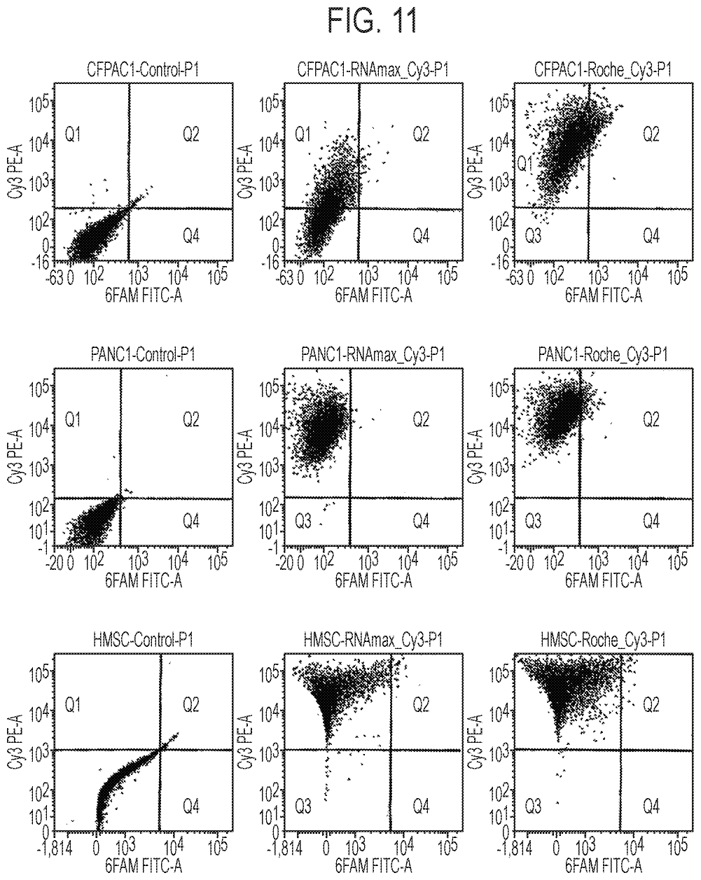

FIG. 11 is a set of plots that illustrate various methods for loading of potential agents for retarding tumor growth transfected directly in vitro into hMSCs and other cells, according to various embodiments. Two transfection reagents were used and compared to a control with no transfection reagent. The siRNA tested for transfection was Mission siRNA which is made fluorescent using Cyanine 3 (called Cy3 hereinafter and designated Cy3 PE-A on the plots). To aid in distinguishing cells, they were also stained with fluorescein (designated 6FAM FITC-A on the plots). Each of the nine plots in FIG. 11 has a logarithmic vertical axis that indicates the intensity of fluorescence from Cy3 and a logarithmic horizontal axis that indicates the intensity of fluorescence from fluorescein. It is not necessary to read the scales on these axis, as becomes evident in the following. Each plot is a scatter plot of the distribution of Cy3 and fluorescein inside cells of one group.

Three cell types were compared, hMSCs on the bottom row of FIG. 11, and the two pancreatic cancer cell lines, PANC-1 on the middle row and CFPAC-1 on the top row. The left column shows the control for the three cell types—that is the distribution of Cy3 and fluorescein when bathed in a 100 nM solution of Mission siRNA tagged by Cy3 without transfection reagents. Most of the cells have low values for Cy3 and fluorescein that define a lower left quadrant Q3 that is marked on each plot. The lower left quadrants are different for each cell type as evident from comparing the three plots in the left column for the control case.

The middle column illustrates the effect of bathing the cells in the 100 nM solution of Mission siRNA tagged by Cy3 when a Lipofectamine RNAiMAX transfection reagent is used. The fluorescein intensities stay low, but the Cy3 intensities inside the cells jump out of quadrant Q3 and into quadrant Q1. This shows effective transfection of Mission siRNA into all three cell types, including hMSCs on the bottom row. A similar result occurs when the X-tremeGene siRNA transfection reagent (available from Roche Molecular Systems Inc., Branchburg, N.J.) is used, as illustrated on the right column for all three cell types.

FIG. 11 demonstrates that siRNA are readily loaded into hMSCs in vitro for use as a donor cell in vivo. An average cell yield from a single well of a 24 well plate is 1.9×10⁵ hMSC cells loaded with siRNA. Introducing 10⁵ hMSC cells loaded with an interfering siRNA is expected to be therapeutic when contacted in vivo with a tumor of syncytial cancer cells.

In other embodiments, other mechanisms are used to transfect siRNA into hMSCs in vitro; including without limitation, using calcium phosphate, or electroporation, or cell squeezing, or by mixing a cationic lipid with the material to produce liposomes, which fuse with the cell membrane and deposit their cargo inside, alone or in some combination.

### 3.4 hMSC can Survive Loading with Certain siRNA that Retard Tumor Growth

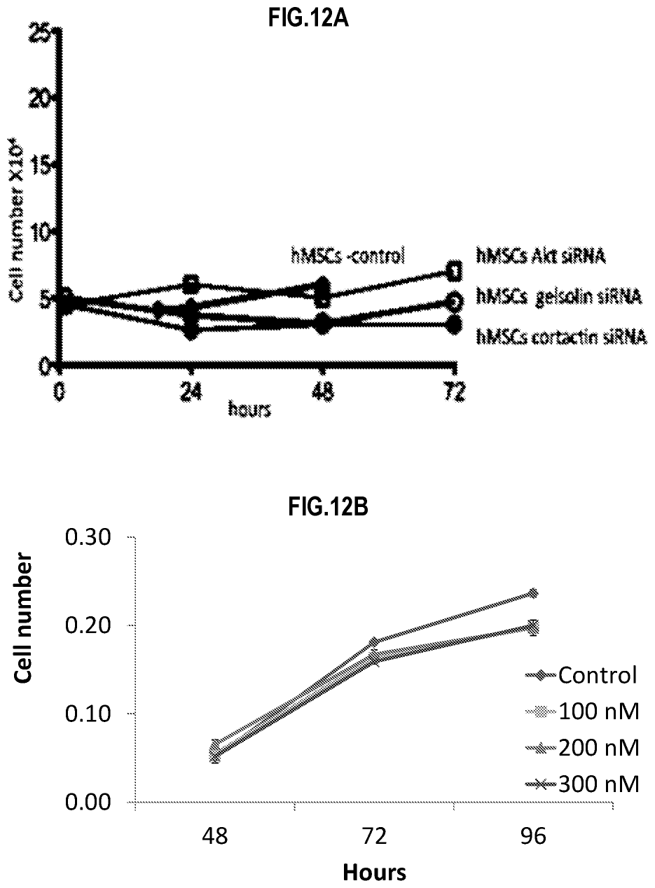

FIG. 12A and FIG. 12B are plots that illustrate survival of hMSCs after loading by potential agents for retarding tumor growth transfected directly in vitro into hMSCs, according to an embodiment. Direct transfection experimental protocols were used, with n=4 experiments for each trace. FIG. 12A is a graph that illustrates example survival of hMSCs when loaded with inhibitory oligonucleotide shown above to reduce the proliferation of at least some cancer cells. The horizontal axis indicates time in hours after transfection and the vertical axis indicates cell number in tens of thousands of cells for each trace the initial population at 0 hours is 50,000 cells. The trace labeled “hMSCs—control” shows the population changes up to 48 hours but does not deviate more than about 10% from the initial population Traces are also shown for hMSCs loaded with Akt siRNA, Gelsolin siRNA, and Cortactin siRNA, respectively. All vary slightly over 72 hours but all end within about 40% of the initial population of hMSCs, sufficient amounts to form a substantial number of gap junctions with neighboring cells.

FIG. 12B is a graph that illustrates example survival of hMSCs when loaded with siRNA mimics for miR-16 shown above to be the most effective agent to reduce the proliferation of all tested cancer cells. The horizontal axis indicates time in hours after loading and the vertical axis indicates cell number in arbitrary units. The trace labeled “Control” shows the population grows up to 96 hours for hMSCs that are not loaded with SiRNA mimics for miR-16. Traces are also shown for hMSCs loaded with siRNA mimics for miR-16 in solutions at different concentration levels of 100 nM, 200 nM and 300 nM, respectively, and labeled accordingly. Each point is the mean of 4 experiments, and the standard deviations, represented by vertical lines, are on the order of the symbol size so are not easily observed. All traces for hMSCs loaded with miR-16 mimics show growth only slightly below the control trace, with little difference among the three non-control traces.

Thus, hMSCs loaded with miR-16 mimics should survive sufficiently long to form gap junctions with target cells and deliver their miR-16 mimic loads to the target cells.

### 3.5 hMSC Forms Functional Gap Junctions with Syncytial Cancer Cells and thus can Transfect Via Gap Junction Certain siRNA that Retard Tumor Growth into Syncytial Cancer Cell

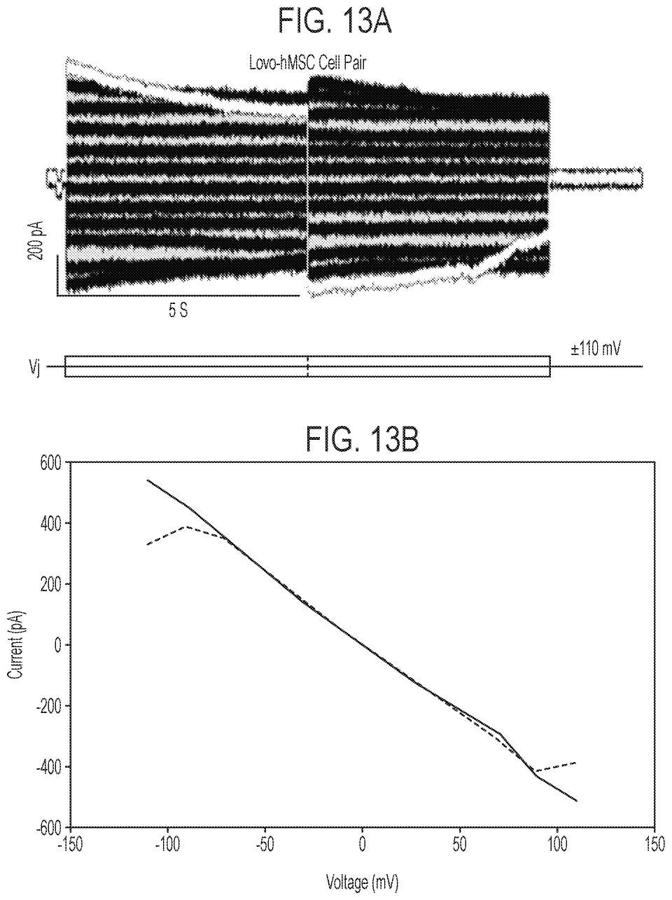

FIG. 13A and FIG. 13B are plots that illustrate formation of gap junctions between an hMSC and a syncytial cancer cell, according to an embodiment. In this case the syncytial cancer cell is a member of the LoVo (Human colon adenocarcinoma) cell line. FIG. 13A shows data resulting from a dual whole cell patch clamp. The horizontal axis is time, and there are two vertical scales, the lower scale shows applied voltage and the upper scale shows resulting current. At zero time there is zero applied voltage and the current is zero. The voltage is immediately lowered to −110 milliVolts (mV, 1 mV=10⁻³ volts) and the current given by the white trace jumps to over 600 picoAmperes (pA, 1 pA=10⁻¹² amperes) then decays slightly. After 5 seconds the voltage is switched to +110 mV and the current reverses to below −600 pA then gradually decays. After ten seconds the voltage returns to zero and the current does as well. In addition voltages between 0 and 110 mV were tested in 10 mV increments to produce the other traces. Current applied to the hMSC was detected in the LoVo cell. Such a response indicates that gap junctions have been formed which pass ions between the cells to carry the measured currents. FIG. 13B is a graph that illustrates a voltage current response curve showing the junctional current in the hMSC cell as a function of the voltage applied to the LoVO cancer cell. Where the current levels off in the dashed line, the coupling is too high to detect the dependence. The solid trace is the measured values, the dashed trace is what would be expected if the conductance between the cells was lower (i.e. the voltage dependence of the conductance was capable of being observed).

### 3.6 Syncytial Cancer Cells Form Functional Gap Junctions with each Other and thus can Transfect Via Gap Junction Certain Sirna that Retard Tumor Growth

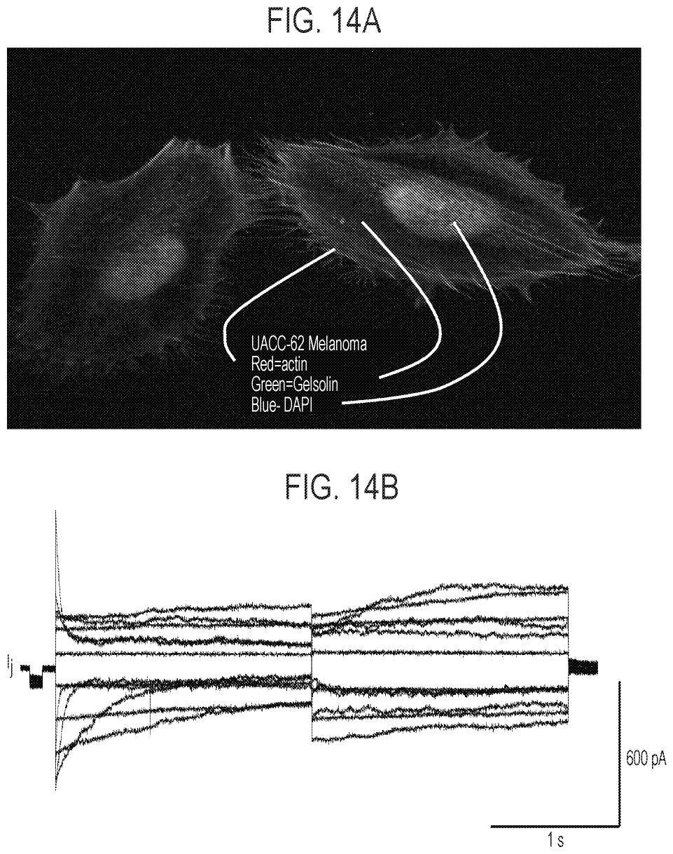

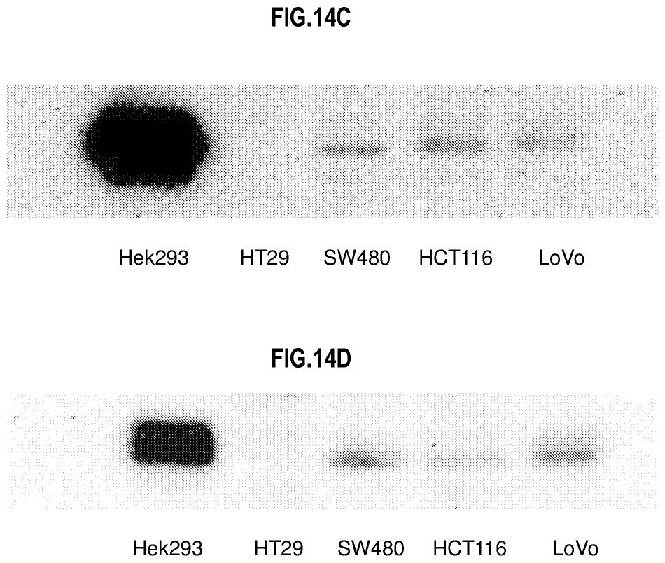

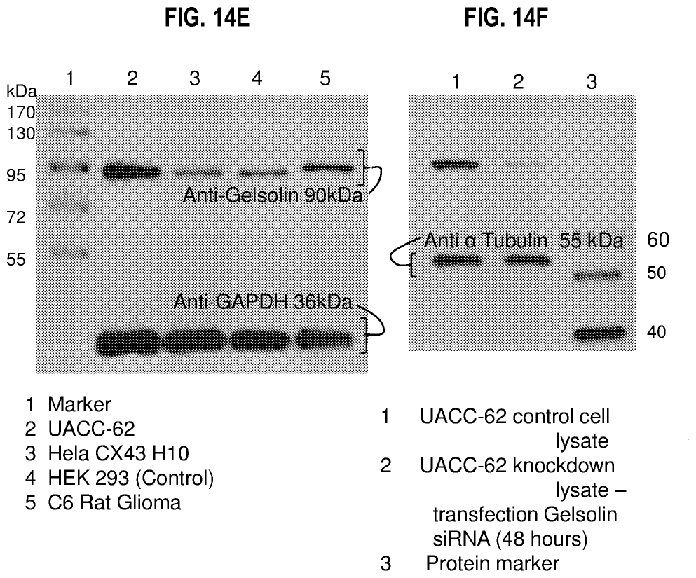

FIG. 14A and FIG. 14B illustrate example formation of gap junctions between two syncytial cancer cells for use in propagating an inhibitory oligonucleotide through multiple cells of a syncytial cancer tumor, according to an embodiment. FIG. 14A is a micrograph of two UACC-62 melanoma cells that have been fluorescently labeled to highlight actin in the outer membrane with a red fluorophore, to highlight DNA with a blue DAPI fluorophore in the nucleus of the cells, and to highlight Gelsolin with a green fluorophore in the cytoplasm. FIG. 14B shows data resulting from a dual whole cell patch clamp. Current applied to one cell was detected in the other.

FIG. 14C and FIG. 14D are images of electrophoresis gels that illustrate gap junctions connexins are found in a variety of colorectal cancer cell lines, for use in various embodiments. In two different experiments, Cx43 was found in Hek293 as a control and in three of four colorectal cell lines including LoVo, human colorectal carcinoma cell line HCT116, and colon adenocarcinoma SW480. Only human colorectal adenocarcinoma cell line HT29 did not express Cx43. The prevailing presence of Cx43 is useful for forming gap junctions with other members of the cell line as well as with hMSCs.

### 3.7 Syncytial Cancer Cells do Transfect Certain siRNA to each Other

FIG. 14E and FIG. 14F are images of electrophoresis gels (Western blots)that illustrate RNA that interfere with the production of several structural or functional proteins are transfected between cancer cells; and, thus represent potential agents for retarding tumor growth, according to an embodiment. FIG. 14E depicts five columns of gel. The first column includes marker molecules of known sizes, including 55 kDa, 72 kDa, 95 kDa, 130 kDa, and 170 kDa. Columns 2 through 5 show the presence of proteins detected by their antibodies (Anti-Gelsolin at 90 kDa, and Anti-GAPDH at 36 kDa) successfully transfected into cells of UACC-62 (malignant melanoma), Hela CX43 H10 (cervical cancer), HeK293 as a control, and C6 (rat Giloma) cell lines, respectively FIG. 14F depicts three columns of gel aligned by size with the image of FIG. 14E. In FIG. 14F, the third column includes marker molecules of known sizes, including 40 kDa, 50 kDa, and 60 kDa. Columns 1 and 2 show the presence of proteins detected by their antibodies (Anti-Gelsolin at 90 kDa, and Anti-α-Tubulin at 55 kDa) down regulated by siRNAs that were transfected successfully into the UACC-62 cells. The proteins knocked down are those shown to be at lower density in the Western blots on the right (gelsolin). The siRNA for gelsolin was transfected in and the level of the protein was reduced. These plots indicate such siRNA can be transfected among coupled tumor cells and thus could be used at least against tumors represented by such cell lines in various embodiments.

### 3.8 Syncytial Cancer Tissue in vitro Delivers siRNA through Multiple Cell Widths of Syncytial Cancer Cells

The determination of gap junction transfection of siRNA from one cancer cell to another is made using scrape loading. In scrape loading, a monolayer of adherent cells are scraped or scratched along a single line in the presence of a gap junction permeable tracer, which becomes incorporated by cells along the scrape, presumably as a result of some mechanical perturbation of the membrane. As normal membrane permeability is re-established, the tracer becomes trapped within the cytoplasm and, with time, may move from the loaded cells into adjacent ones connected by functional gap junctions made of connexin channels. The distance at which the fluorescent dye diffuses during a certain period away from the scrape line is indicative of gap junction intercellular communication.

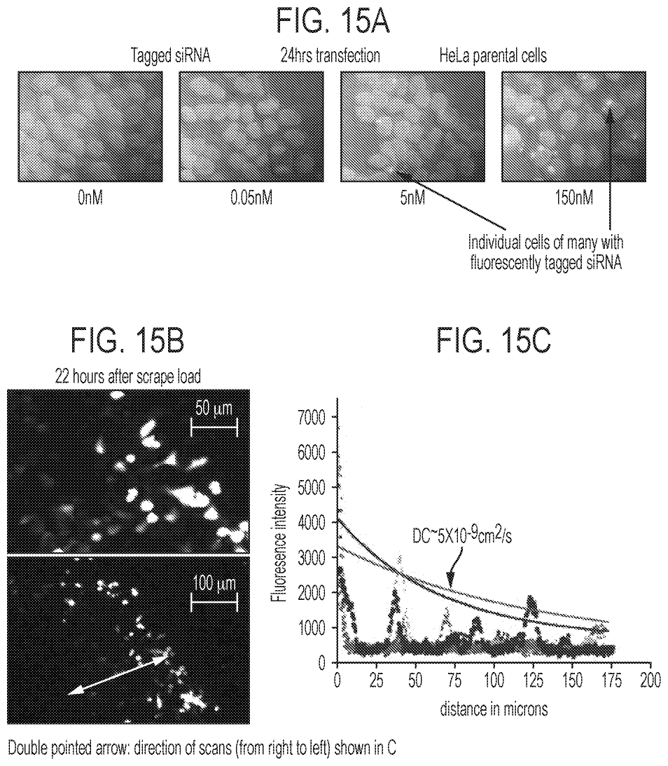

FIG. 15A through FIG. 15C are images and plots that illustrate propagation of siRNA through multiple cells of a syncytial cancer tumor, according to an embodiment In FIG. 15A, the amount of fluorophore tagged siRNA loaded into HeLa cervical cancer cell line cells is shown in four images for four different concentration of the tagged siRNA in solution, at 0 nM, 0.05 nM, 5 nM and 150 nM respectively, each after 24 hours of transfection. At higher concentrations, more cells are transfected with bright dots of fluorescently tagged siRNA.

FIG. 15B shows images of a HeLa cervical cancer monolayer 22 hours after a scrape load event that cut diagonally from top center to lower right corner. The top image is a portion of the lower image at twice the magnification. The cells along the scrape line are bright with tagged siRNA, while adjacent cells also show some tagged siRNA due to gap junctions transfer. The double arrow line segment shows a direction perpendicular to the scrape direction, where data are plotted in FIG. 15C.

FIG. 15C is a graph that illustrates fluorescence intensity dropping with distance from a scrape load line. The horizontal axis is distance in microns; and, the vertical axis is fluorescence intensity in arbitrary units. The fluorescent dye concentrates in the nucleus of each cell, forming a peak. The number of cell widths through which this labeled siRNA is transported can be determined by counting the peaks. The fluorescence intensity falls to about 5% of the scrape line value at a range of about 175 microns, corresponding to about 11 cell widths. Thus the tagged siRNA was propagated by gap junctions through ten intervening cells to reach the last cell showing the tagged siRNA. A fit of a diffusion curve produces a diffusion constant, D_{C}, of about 5×10⁻⁹ squared centimeters per second.

This demonstrates that inhibitory oligonucleotide can be expected to propagate via gap junction from one cancer cell to another after introduction by the loaded hMSC, multiplying the effect of the hMSCs that are usually outnumbered by the cancer cells.

### 3.9 Co-Culture In Vitro with Loaded hMSC does Retard Tumor Growth

When loaded with an appropriate inhibitory oligonucleotide, hMSCs co-cultured in vitro with cancer cells are discovered to lead to reduction of tumor growth. This is in stark contrast to the result obtained (e.g., in FIG. 3A and FIG. 3B) when inhibitory oligonucleotide is not loaded into the hMSCs. Based on the above experiments, it is believed that this result is due to transfection of the inhibitory oligonucleotide via gap junctions from the hMSCs to adjacent cancer cells and from those adjacent cancer cells on to successively more distant cancer cells.

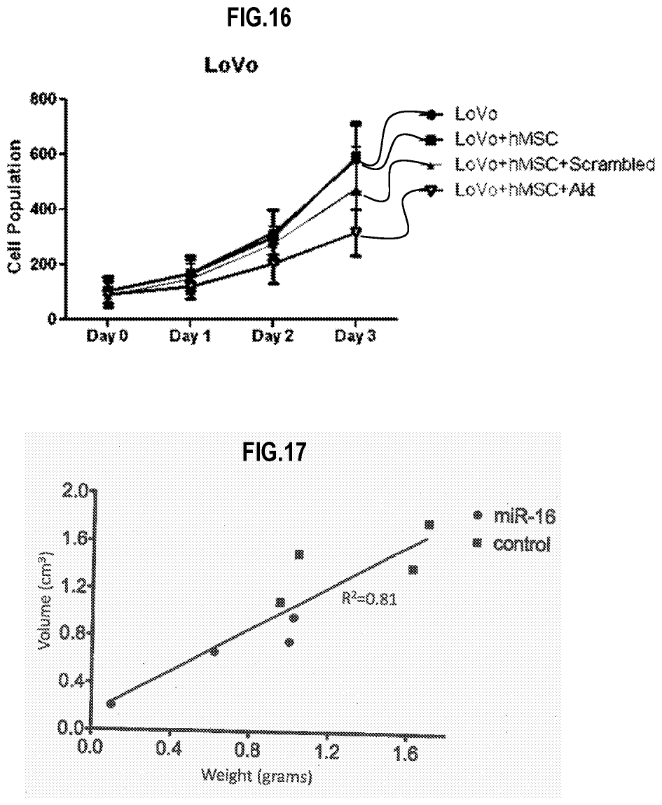

FIG. 16, is a plot that illustrates relative effects on colorectal tumor growth by co-culture with hMSCs in vitro; according to an embodiment. This experiment used the LoVo colorectal cell line. Indirect transfection experimental protocols were used, with n=15 experiments for each trace. The horizontal axis indicates time in days; and, the vertical axis indicates cell population in arbitrary units. Traces are shown for LoVo cells cultured alone and for LoVo cells cultured with hMSCs that were not loaded with inhibitory oligonucleotide. A trace is also shown for LoVo cells co-cultured with hMSCs that had been loaded with Akt siRNA (labeled “LoVo+hMSC+Akt”). A trace is also shown for LoVo cells co-cultured with hMSCs that had been loaded with scrambled siRNA (labeled “LoVo+hMSC+Scrambled”). A scrambled siRNA is a siRNA whose sequence is not complementary to any known gene sequence and therefore represents a nonsense siRNA. Each trace plots the mean of 15 cultures with the standard deviation indicated by vertical line segments. On day 3, LoVo+hMSC+Akt was significantly better (p<0.001) than all three other groups. LoVo+hMSC+Scrambled was significantly better (p<0.05) than LoVo alone and LoVo+hMSC.

FIG. 17 is a plot that illustrates direct relationship between tumor weight and tumor volume for comparing various remaining plots. The horizontal axis indicates weight in grams; and, the vertical axis indicates volume in cubic centimeters (cm³). Data is based on both control tumors and tumors transfected with miR-16 mimics for the smaller tumors that were weighed. The relationship is linear for weights from about 0.1 grams to about 1.6 grams with an R²=0.81.

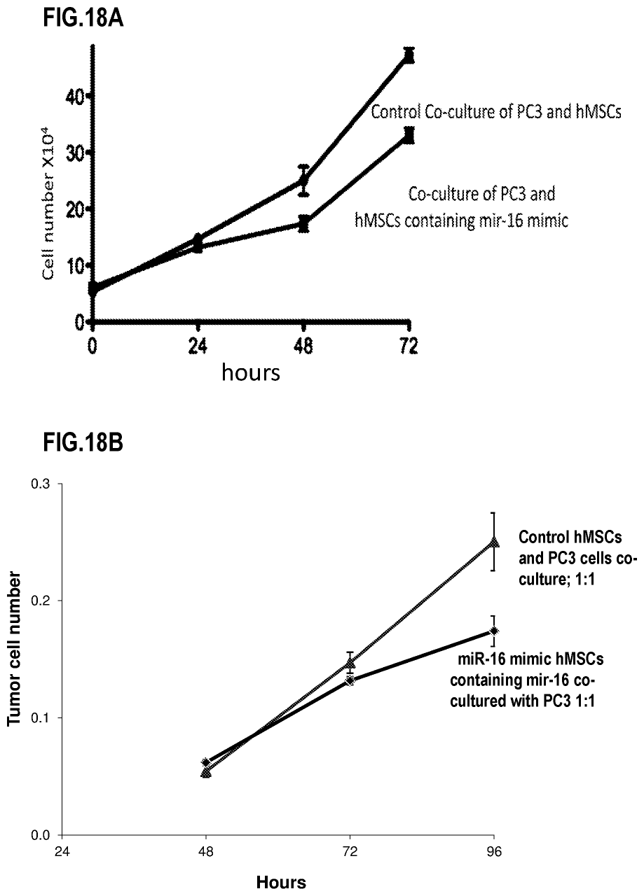

FIG. 18A and FIG. 18B are plots that illustrate relative effects on prostate tumor growth by co-culture in vitro with hMSCs loaded with miR-16 or a siRNA mimic for miR-16; according to an embodiment. Indirect transfection experimental protocols were used, with n=4 experiments for each trace. FIG. 18A is a graph that illustrates data from one example set of experiments. The horizontal axis indicates time in hours; and, the vertical axis indicates cell number in tens of thousands of cells. The control trace shows the effects of co-culturing PC-3 prostate cell line with hMSCs not loaded with inhibitory oligonucleotide. The other trace shows the effects of co-culturing the PC-3 prostate cell line with hMSCs loaded with a miR-16 mimic Each trace plots the average of four cultures and the standard deviation is indicated by vertical line segments. There is about a 25% reduction in tumor growth rate; not quite as dramatic as direct transfection of miR-16 mimics It is anticipated that longer culturing times would show more reductions as the number of gap junctions increase and more miR-16 mimic is delivered to the PC-3 cells.

FIG. 18B is a graph that illustrates data from another example set of experiments that extend a day longer, to 96 hours. The horizontal axis indicates time in hours; and, the vertical axis indicates tumor cell number in units of absorbance. The control trace shows the effects of co-culturing PC-3 prostate cell line with hMSCs not loaded with inhibitory oligonucleotide in an even mix of cells of each type (1:1). The other trace shows the effects of co-culturing the PC-3 prostate cell line with hMSCs loaded with a miR-16 mimic, also in a 1:1 mix. The hMSCs were loaded in a solution of 150 nM miR-16 mimic. Each trace plots the average of six cultures and the standard deviation is indicated by vertical line segments. There is about a 30% to 40% reduction in tumor growth rate; somewhat better than obtained at 72 hours here and at 72 hours in FIG. 18A.

### 3.10 Treatment In Vivo with Loaded hMSC does Retard Tumor Growth

In this section, it is demonstrated that hMSCs loaded with inhibitory oligonucleotide does reduce tumor growth in vivo, in stark contrast to the results shown in FIG. 3A and FIG. 3B for hMSCs not loaded with inhibitory oligonucleotide.

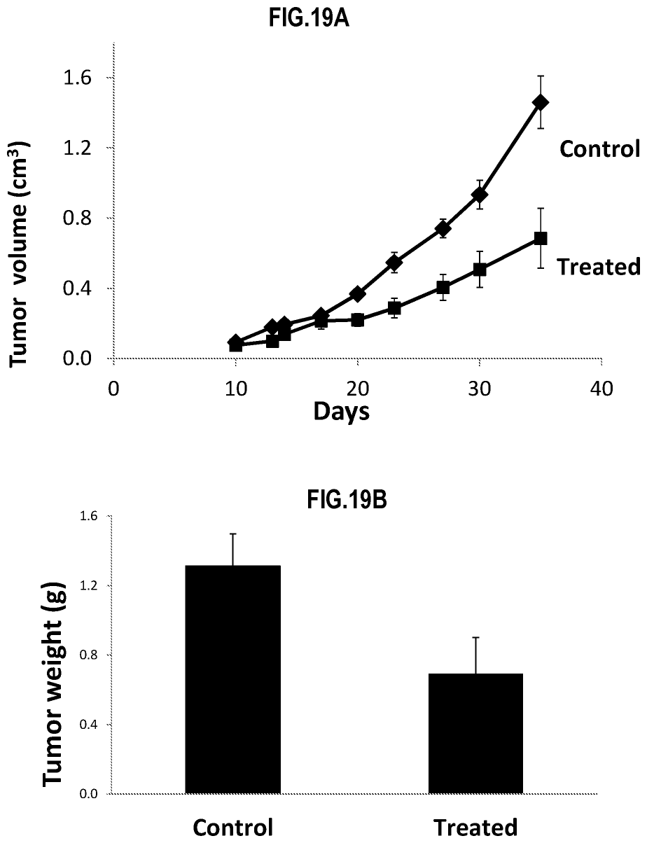

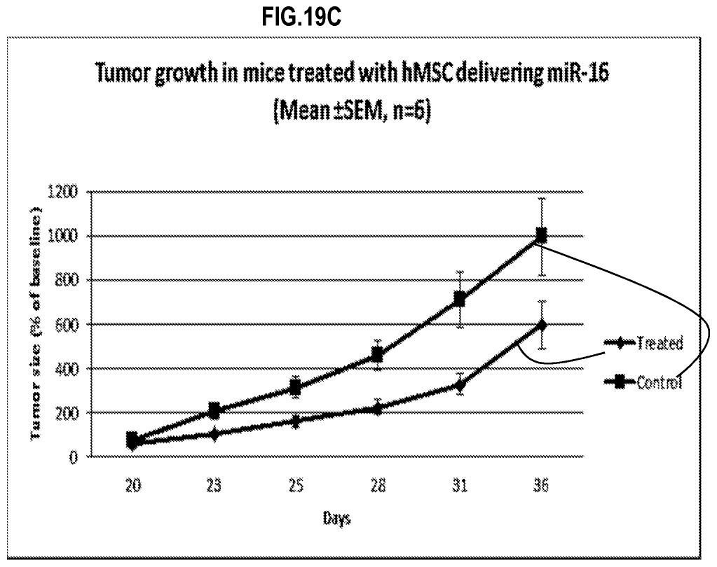

FIG. 19A through FIG. 19C are plots that illustrate effect on prostate tumor growth by in vivo treatment with hMSCs loaded with miR-16 mimic; according to an embodiment. FIG. 19A is a graph that illustrates example reduction in tumor growth in vivo using hMSCs loaded with miR-16 mimic The horizontal axis indicates time in days, out to 40 days, and the vertical axis indicates the tumor volume in cubic centimeters (cm³). At day zero, 1 million cells of the prostate cancer PC-3 cell line were injected into each nude mouse in two groups of nude mice, n=4 mice in each group. At day ten, hMSCs, loaded with miR-16 mimic in a 100 nM solution, were injected into the tumor for each nude mouse in one of the groups. The control trace indicates the average tumor volume of the untreated group of nude mice that did not receive the hMSCs loaded with miR-16 mimic, with the standard deviation indicted by vertical lien segments. The treated trace indicates the average tumor volume of the treated group of nude mice that did receive the hMSCs loaded with miR-16 mimic, again with the standard deviation indicated by vertical line segments. At 35 days, the treated group tumor volume is about 50% of the tumor volume of the untreated, control group. The difference is statistically significant and therapeutically important.

FIG. 19B is a bar graph that illustrates tumor weight for tumor cells grown alone (control) and in the presence of hMSCs that are loaded with siRNA that mimics miR-16 at 35 days when the animals are sacrificed from the experiments depicted in FIG. 19A. The horizontal axis indicates the group and the vertical axis indicates weight in grams. The first bar indicates the average weight of four tumors in the control group and the solid bar the avenge weight of four tumors in the group grown with hMSCs, the vertical line segments indicate the standard error of the mean (SEM). There is a significant decrease in tumor growth associated with treatment by hMSCs that are loaded with miR-16 mimic This result should be contrasted with the result depicted in FIG. 3B.

FIG. 19C is a graph that illustrates example reduction in tumor growth in vivo using hMSCs loaded with miR-16 mimic in terms of tumor size (width and height). The horizontal axis indicates time in days, out to 36 days, and the vertical axis indicates the tumor size in terms of % of a baseline size. At day zero, 1 million cells of the prostate cancer PC-3 cell line were injected into each nude mouse in two groups of nude mice, with 6 mice in each group. At day 14, one million hMSCs, loaded with siRNA serving as miR-16 mimic, were injected into the tumor for each nude mouse in one of the groups. The control trace indicates the average tumor size of the untreated group of nude mice that did not receive the hMSCs loaded with miR-16 mimic, with the standard error of the mean (SEM) indicted by vertical lien segments. The treated trace indicates the average tumor size of the treated group of nude mice that did receive the hMSCs loaded with miR-16 mimic, again with the SEM indicated by vertical line segments. At 36 days, the treated group tumor size is about 60% of the tumor size of the untreated, control group. The difference is statistically significant and therapeutically important.

Based on the experimental results for one cancer cell line, and the parallels observed in connexins occurrence, gap junction formation, and responses to miR-mimics for other syncytial cancer cell lines, it is anticipated that similar significant and therapeutic reductions in tumor growth can be obtained for other cancers, including the cancers illustrated herein (cervical cancer, colorectal cancer, melanoma, pancreatic cancer, prostate, non-small cell lung cancers, and rat Giloma) by injecting or otherwise contacting tumors with hMSCs that have been loaded with inhibitory oligonucleotide, such as miR-16 mimics, Cortactin siRNA, Gelsolin siRNA, Akt siRNA, and miR-34a mimics, among others.

### 4. Alternatives, and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items. elements or steps. Furthermore, the indefinite article “a” or “an” is meant to indicate one or more of the item, element or step modified by the article.

### REFERENCES

Tian et al., 2011, “Human mesenchymal stem cells play a dual role on tumor cell growth in vitro and in vivo,” J Cell Physiol., v226(#7), pp 1860-7, 2011, doi: 10.1002/jcp.22511.

Plotnikov et al., 2003, “Human mesenchymal stem cells transfected with HCN2 as a gene delivery system to induce pacemaker function in canine heart,” Circulation, v108: IV-547.

Valiunas et al., 2002, “Cardiac gap junction channels show quantitative differences in selectivity.” Cir. Res., v91, pp 104-111.

1. A method of treating cancer in vivo, the method comprising:
a) introducing in vitro into a plurality of human mesenchymal stem cells (hMSCs) at least one type of inhibitory oligonucleotide; and
b) contacting a tumor tissue comprising a plurality of syncytial cancer cells with the plurality of hMSCs in vivo under conditions permitting a hMSC of the plurality of hMSCs to form a gap junction channel with a first syncytial cancer cell of the tumor tissue, whereby the at least one type of inhibitory oligonucleotide is delivered into the first syncytial cancer cell by traversing the gap junction channel and the at least one type inhibitory oligonucleotide is delivered into a second syncytial cancer cell of the tumor tissue by traversing a gap junction channel between the first syncytial cancer cell and the second syncytial cancer cell.

2. The method as recited in claim 1, wherein the at least one type of inhibitory oligonucleotide does not kill the hMSC before the hMSC can deliver the at least one type of inhibitory oligonucleotide to the first syncytial cancer cell.

3. The method as recited in claim 1, wherein the plurality of hMSCs comprises about 10⁵ hMSCs.

4. The method as recited in claim 1, wherein the at least one type of inhibitory oligonucleotide is selected from a group comprising miR-16, miR-34a, siRNA that mimics miR-16, siRNA that mimics miR-34a; siRNA that interferes with translation of Cortactin, siRNA that interferes with translation of Akt, siRNA that interferes with translation of Gelsolin, siRNA that interferes with translation of a-Tubulin, siRNA that interferes with translation of GAPDH, and siRNA that interferes with translation of Kras^{GAT}.

5. The method as recited in claim 1, wherein the tumor tissue is a member of a group comprising cervical cancer tissue, colorectal cancer tissue, melanoma tissue, pancreatic cancer tissue, prostate cancer tissue, non-small cell lung cancers, and rat Giloma.

6. The method as recited in claim 1, wherein the tumor tissue is prostate cancer tissue and the at least one type of inhibitory oligonucleotide is siRNA that mimics miR-16.

7. The method as recited in claim 1, wherein introducing in vitro into the plurality of human mesenchymal stem cells (hMSCs) at least one type of inhibitory oligonucleotide further comprises culturing the plurality of hMSCs in a 20 nanoMole solution of the at least one type of inhibitory oligonucleotide that codes for the at least one type of inhibitory oligonucleotide.

8. The method as recited in claim 1, wherein introducing in vitro into the plurality of human mesenchymal stem cells (hMSCs) at least one type of inhibitory oligonucleotide further comprises culturing the plurality of hMSCs in a solution of a transfection reagent and the at least one type of inhibitory oligonucleotide that codes for the at least one type of inhibitory oligonucleotide.
